(19) United States
(12) Patent Application Publication
(10) Pub. No.: US 2021/0393350 A1
(43) Pub. Date: Dec. 23, 2021

(54) PARALLEL LINK DEVICE, MASTER-SLAVE SYSTEM, AND MEDICAL MASTER-SLAVE SYSTEM

(71) Applicant: SONY CORPORATION, TOKYO (JP)

(72) Inventors: KAZUO HONGO, TOKYO (JP); KIYOKAZU MIYAZAWA, TOKYO (JP)

(21) Appl. No.: 17/288,627

(22) PCT Filed: Sep. 9, 2019
(86) PCT No.: PCT/JP2019/035424

§ 371 Date: Apr. 26, 2021

(30) Foreign Application Priority Data
Nov. 2, 2018 (JP) 2018-207784

(51) Int. Cl. A61B 34/37; A61B 34/00; B25J 9/00; B25J 9/16; B25J 13/08; B25J 3/00

(52) U.S. Cl. CPC A61B 34/37; A61B 34/70; B25J 3/00; B25J 9/1607; B25J 13/088; B25J 9/0081

(57) ABSTRACT

Provided is a parallel link device that has an RCM structure and can drive translation and rotation independently.

The parallel link device includes: an actuation unit that has a base portion, an end portion, and a plurality of link portions configured to couple the base portion and the end portion and drives the link portion using a first actuator mounted on the base portion to actuate the end portion with respect to the base portion; and a transmission unit that transmits drive of a second actuator mounted on the base portion to a mechanism portion mounted on the end portion along each of at least two of the plurality of link portions.

### TECHNICAL FIELD

The technology disclosed herein relates to a parallel link device, a master-slave system, and a medical master-slave system.

### BACKGROUND ART

A parallel link robot has features that fingertips can be configured very lightly, the robot can be configured at relatively low cost, a selected motor can be arranged on the base and therefore it is unnecessary to drive the own weight and the required performance of the motor can be suppressed, and the like. Therefore, a wide range of robots such as industrial robots and medical robots have attracted attention in recent years.

For example, a medical parallel link device having a remote center of motion (RCM) structure has been developed (see Patent Document 1). Here, the RCM structure is regarded as a structure in which a rotation center (i.e., remote rotation center) is arranged at a position away from the rotation center of a drive mechanism such as a motor to realize pivot (fixed point) motion. The RCM structure is highly safe because it can realize a structure that always passes through the position (e.g., trocar position) of a hole made in the body of a patient during surgery, and has already been adopted in some robots and medical apparatuses. On the other hand, a translation structure is required to adjust the position of a hole. If translation and rotation are structurally independent, it is unnecessary to control the translation structure at the same time during the rotation, which is preferable in that the control calculation becomes easy. Furthermore, unnecessary action of the actuator is reduced, and durability can be improved. However, there are few types of RCM structures in which all motors are fixed to the base and which is configured with a parallel mechanism. Furthermore, it is practically difficult to have a structure in which translation and rotation can be driven independently and both actuators are mounted on the base.

Furthermore, although a positioning system for a surgical instrument has been developed that includes a parallel mechanism realizing an RCM structure by combining a plurality of delta structures (see Patent Document 2), there is a problem that the lateral width of the parallel link becomes wide since the delta structure is used. Furthermore, although a support arm device that realizes a pivot with a small occupied area by combining link structures has also been developed (see Patent Document 3), translation drive is impossible.

### CITATION LIST

### Patent Document

Patent Document 1: WO2014/108545
Patent Document 2: WO2012/020386
Patent Document 3: WO2017/077755
Patent Document 4: Japanese Patent Application Laid-Open No. 2004-261886
Patent Document 5: Japanese Patent Application Laid-Open No. 2005-144627
Patent Document 6: Japanese Patent Application Laid-Open No. 2016-223482

### SUMMARY OF THE INVENTION

### Problems to be Solved by the Invention

The technology disclosed herein has been made in consideration of the above problems, and an object thereof is to provide a parallel link device, a master-slave system, and a medical master-slave system that have an RCM structure, can independently drive translation and rotation, and has a structure in which all actuators are mounted on the base.

### Solutions to Problems

The first aspect of the technology disclosed herein is a parallel link device including:

an actuation unit that has a base portion, an end portion, and a plurality of link portions configured to couple the base portion and the end portion and drives the link portion using a first actuator mounted on the base portion to actuate the end portion with respect to the base portion; and

a transmission unit that transmits drive of a second actuator mounted on the base portion to a mechanism portion mounted on the end portion along each of at least two of the plurality of link portions.

For example, the mechanism portion has two rotational degrees of freedom. Then, the transmission unit is configured to transmit the drive of the second actuator along each of two of the plurality of link portions to rotate the mechanism portion around each axis.

Alternatively, the mechanism portion has three rotational degrees of freedom. Then, the transmission unit is configured to transmit the drive of the second actuator along each of three of the plurality of link portions to rotate the mechanism portion around each axis.

Alternatively, the mechanism portion includes a spherical parallel link having three rotational degrees of freedom configured to move on a spherical surface including a common center. Then, the transmission unit is configured to transmit the drive of the second actuator along each of three of the plurality of link portions to rotate the mechanism portion around each axis.

Furthermore, a sensor that measures the posture, acceleration, angular acceleration, or the like of the mechanism portion may be further provided.

Furthermore, the second aspect of the technology disclosed herein is a master-slave system including a master device and a slave device remotely operated by the master device,

in which the slave device includes:

an actuation unit that has a base portion, an end portion, and a plurality of link portions configured to couple the base portion and the end portion and drives the link portion using a first actuator mounted on the base portion to actuate the end portion with respect to the base portion; and

a transmission unit that transmits drive of a second actuator mounted on the base portion to a mechanism portion mounted on the end portion along each of at least two of the plurality of link portions.

However, the term “system” used here refers to a logical collection of a plurality of devices (or functional modules that implement a specific function), and whether the devices or the functional modules are located in a single housing or not does not matter (the same applies hereinafter).

Furthermore, the third aspect of the technology disclosed herein is a medical master-slave system including:

a master device that accepts operation input to a medical instrument by an operator; and

a slave device that has a base portion, an end portion, and a plurality of link portions configured to couple the base portion and the end portion, holds the medical instrument on the end portion, and receives the operation input to the medical instrument from the master device to control the medical instrument,

in which the slave device includes:

an actuation unit that actuates the end portion with respect to the base portion; and

a transmission unit that transmits drive of a second actuator mounted on the base portion to a mechanism portion mounted on the end portion along each of at least two of the plurality of link portions.

### Effects of the Invention

It is possible with the technology disclosed herein to provide a parallel link device, a master-slave system, and a medical master-slave system that have an RCM structure, can independently drive translation and rotation, and have a structure in which all actuators are mounted on the base.

Note that the effects described herein are merely exemplification, and effects of the present invention are not limited thereto. Furthermore, the present invention may produce additional effects in addition to the above effects.

Other objects, characteristics, or advantages of the technology disclosed herein will further become apparent from the more detailed description based on the embodiments described later or the accompanying drawings.

### BRIEF DESCRIPTION OF DRAWINGS

FIG. 1 is a view (perspective view) illustrating a configuration example of a parallel link device 100.

FIG. 2 is a view (side view) illustrating a configuration example of the parallel link device 100.

FIG. 3 is a view (top view) illustrating a configuration example of the parallel link device 100.

FIG. 4 is a view illustrating the structure of an additional link mechanism portion equipped in addition to a link portion 110.

FIG. 5 is a view illustrating the degree-of-freedom configuration of an RCM structure portion 200.

FIG. 6 is a view illustrating an example in which the parallel link device 100 takes various postures.

FIG. 7 is a view illustrating an example in which the parallel link device 100 takes various postures.

FIG. 8 is a view illustrating an example in which the parallel link device 100 takes various postures.

FIG. 9 is a view illustrating an example in which the parallel link device 100 takes various postures.

FIG. 10 is a view illustrating a configuration example (perspective view) of a parallel link device 1000 according to the second embodiment.

FIG. 11 is a view (perspective view) illustrating a configuration example of a parallel link device 1100.

FIG. 12 is a view (front view) illustrating a configuration example of the parallel link device 1100.

FIG. 13 is a view (top view) illustrating a configuration example of the parallel link device 1100.

FIG. 14 is a view (perspective view) illustrating a configuration example of an RCM structure portion 2000.

FIG. 15 is a view (front view) illustrating a configuration example of the RCM structure portion 2000.

FIG. 16 is a view (top view) illustrating a configuration example of the RCM structure portion 2000.

FIG. 17 is a view (perspective view) illustrating a configuration example of a parallel link device 1700.

FIG. 18 is a view (front view) illustrating a configuration example of the parallel link device 1700.

FIG. 19 is a view (top view) illustrating a configuration example of the parallel link device 1700.

FIG. 20 is a view (perspective view) illustrating a configuration example of an RCM structure portion 3000.

FIG. 21 is a view (front view) illustrating a configuration example of the RCM structure portion 3000.

FIG. 22 is a view (top view) illustrating a configuration example of the RCM structure portion 3000.

FIG. 23 is a view illustrating a configuration example (top view) of the parallel link device 1000 according to the second embodiment.

FIG. 24 is a view illustrating a configuration example (top view) of the parallel link device 1000 according to the second embodiment.

FIG. 25 is a diagram schematically illustrating the functional configuration of a master-slave type robot system 2500.

### MODE FOR CARRYING OUT THE INVENTION

The following description will explain embodiments of the technology disclosed herein in detail with reference to the drawings.

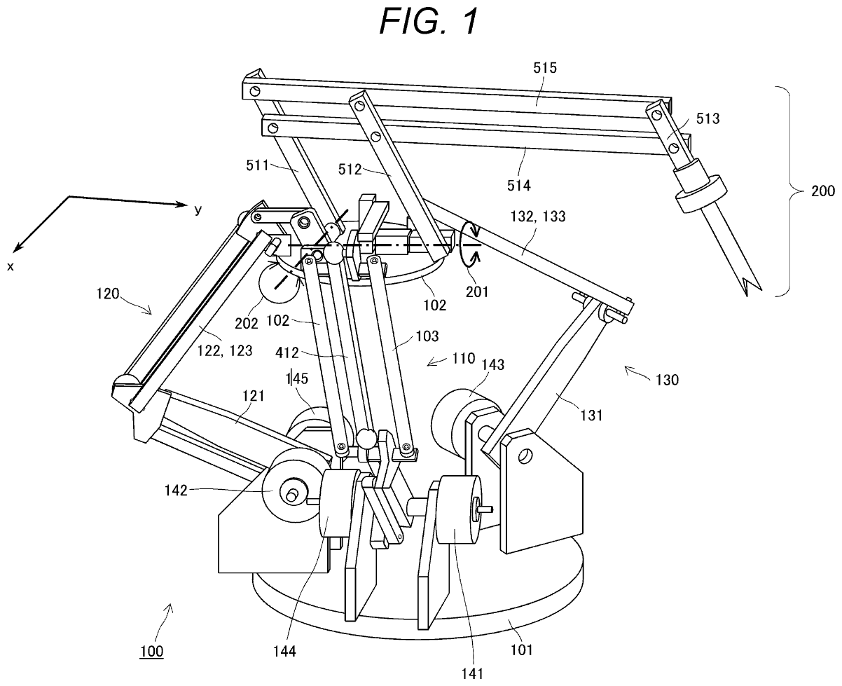

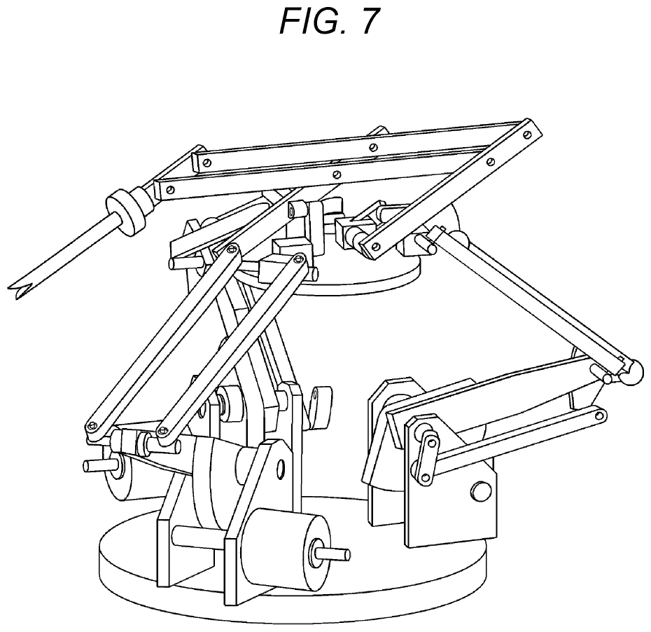

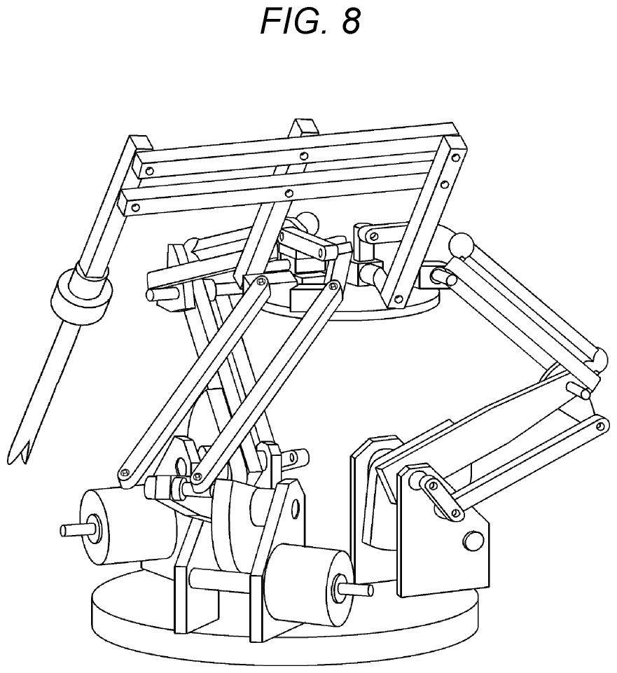

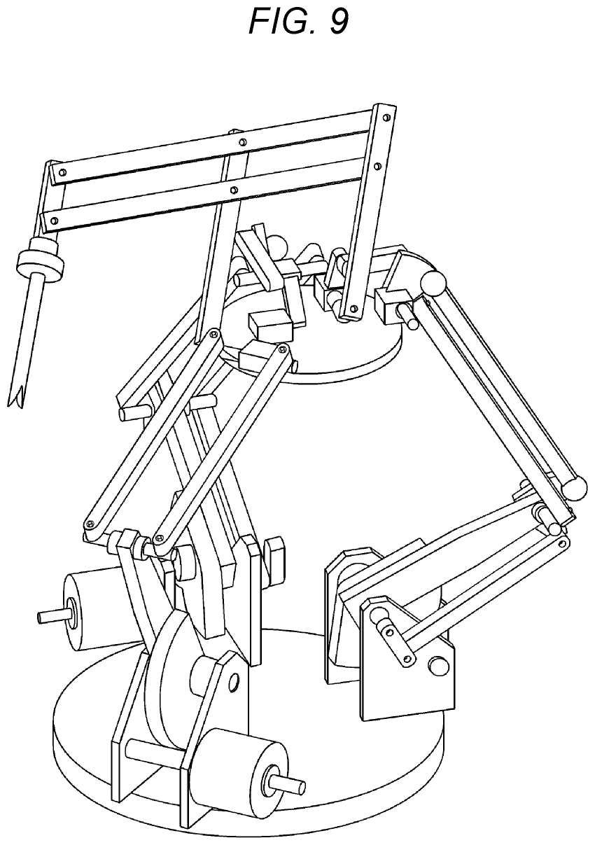

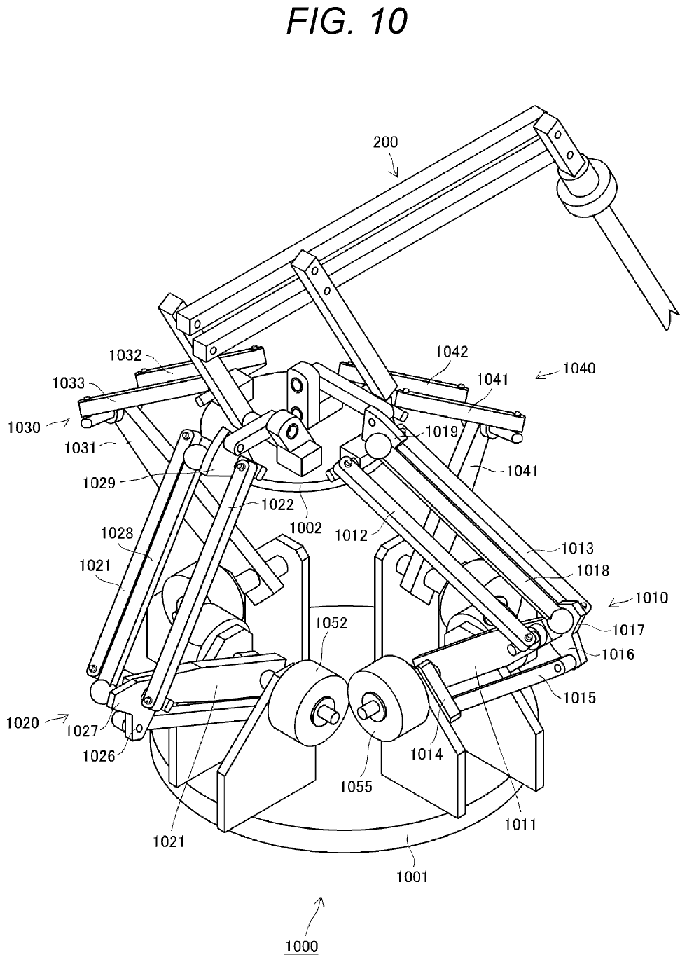

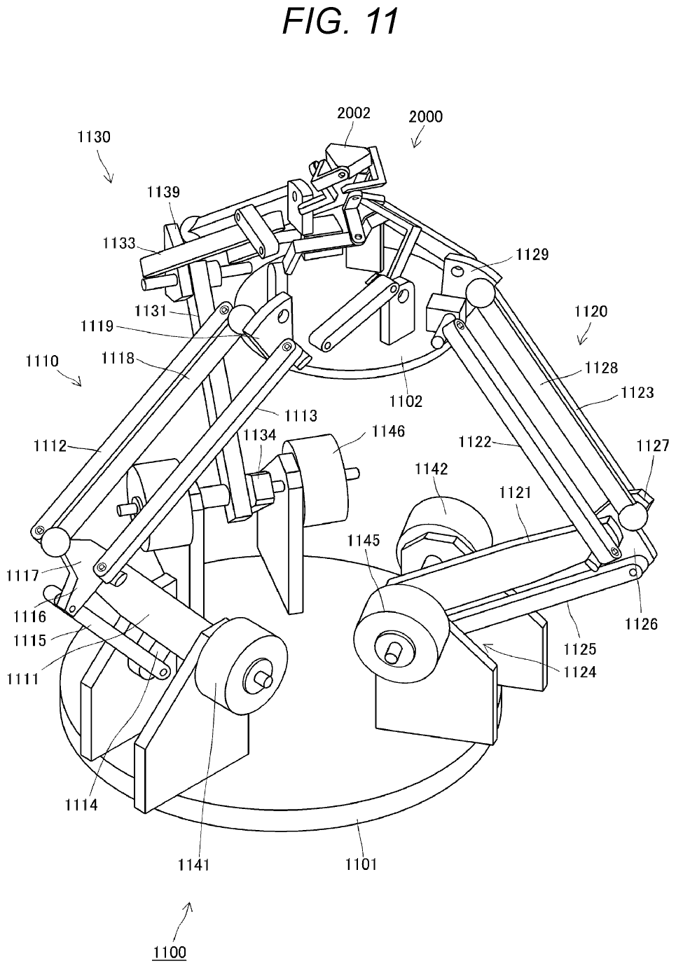

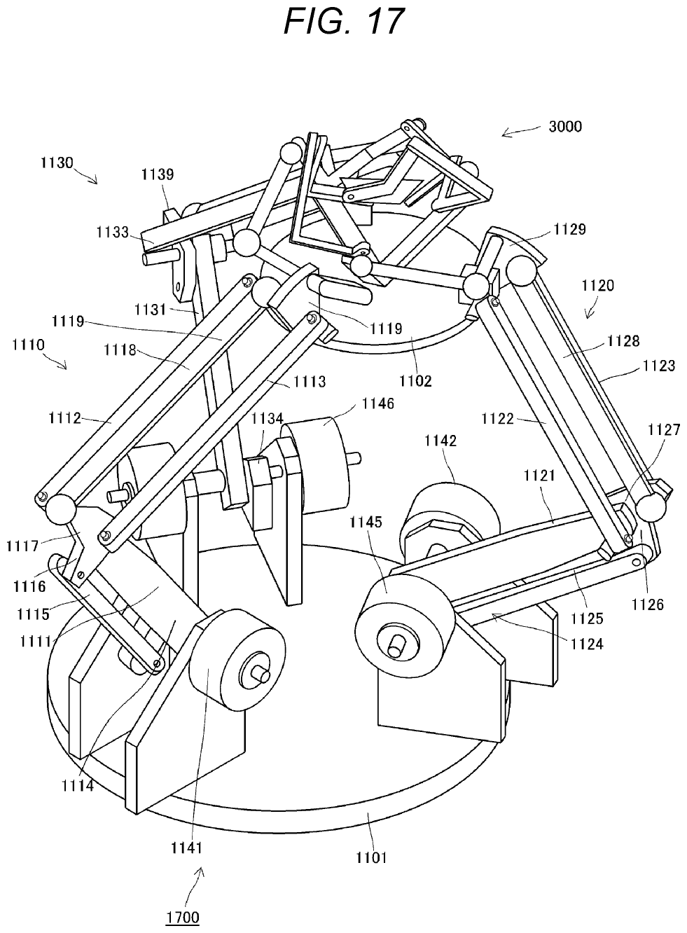

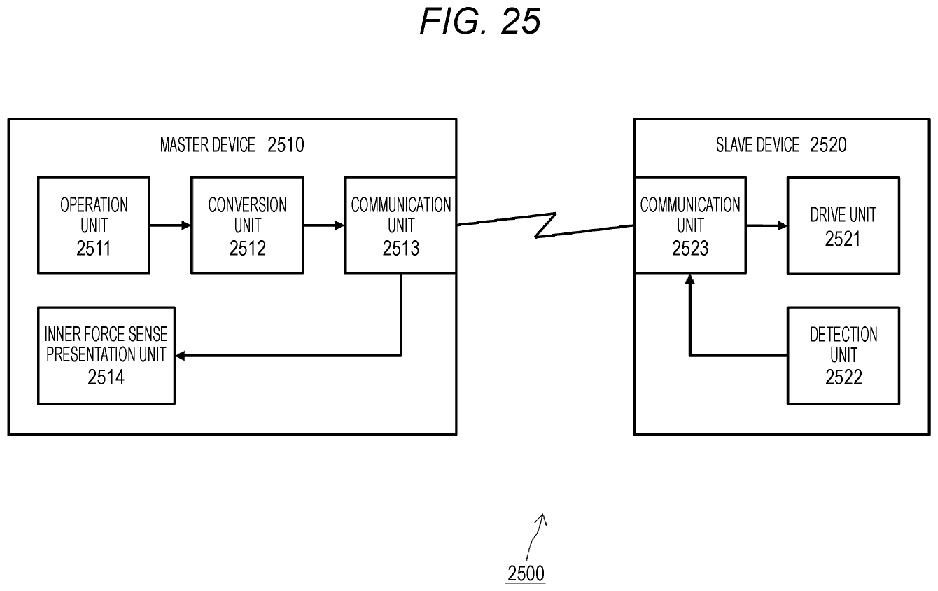

Hereinafter, the structure of a typical parallel link device will be described first as a first embodiment with reference to FIGS. 1 to 9. Then, the structure of a parallel link device according to a variation will be described as a second embodiment with reference to FIG. 10. Further, the structure of a parallel link device according to a further variation will be described as a third embodiment with reference to FIGS. 11 to 16. Further, the structure of a parallel link device according to a further variation will be described as a fourth embodiment with reference to FIGS. 17 to 22. Further, a master-slave type robot system 2500 applied to the slave side of the parallel link device will be described as a fifth embodiment with reference to FIG. 25.

### Embodiment 1

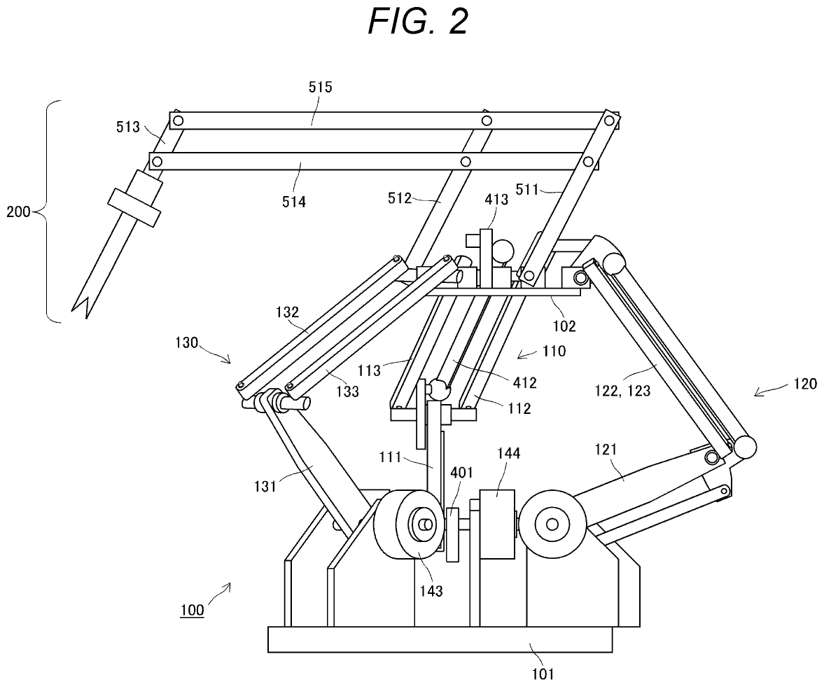

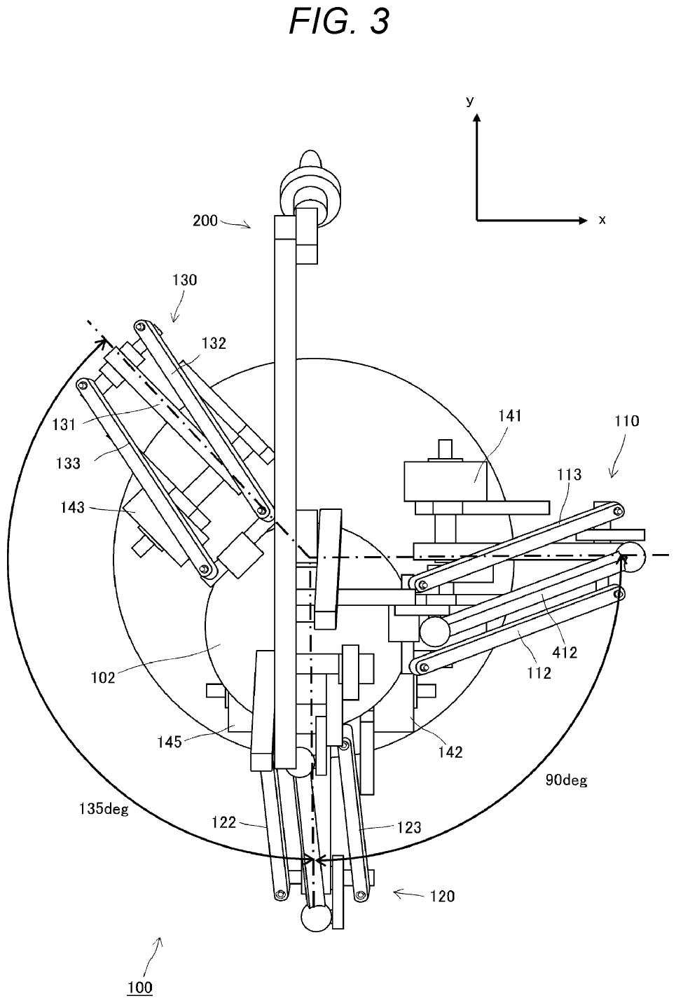

FIGS. 1 to 3 illustrate a configuration example of a parallel link device 100 according to the first embodiment. However, FIG. 1 shows a view of the parallel link device 100 viewed obliquely, FIG. 2 shows a view of the parallel link device 100 viewed from a side, and FIG. 3 shows a view of the parallel link device 100 viewed from above.

The illustrated parallel link device 100 includes a base portion 101, an end portion 102 that translates with respect to the base portion 101, and a plurality of link portions 110, 120, and 130 that is coupled to the base portion 101 and supports the end portion 102, and configures a delta type parallel link that generates three-translational-degree-of-freedom action. Furthermore, five actuators 141 to 145 for driving the link portions 110, 120, and 130 are mounted on the base portion 101. Although the actuators 141 to 145 are attached via a rib-shaped member projecting from the upper surface of the base portion 101 in the example illustrated in FIGS. 1 to 3, note that the actuators are not limited to a specific attachment structure. Moreover, an RCM structure portion 200 that realizes pivot motion is mounted on the end portion 102. The RCM structure portion 200 can act with two degrees of freedom in this embodiment, and the details will be described later.

The link portion 110 includes an upper arm link 111, and a pair of forearm links 112 and 113. One end of the upper arm link 111 is turnably coupled to the base portion 101, and the other end is turnably coupled to the pair of forearm links 112 and 113 via passive joints. Furthermore, the forearm links 112 and 113 support the end portion 102 at the other ends. However, it is preferable to have a structure in which the upper arm link 111 and each of the forearm links 112 and 113, and each of the forearm links 112 and 113 and the end portion 102 are connected by, for example, spherical joints so as to absorb the inclination. Similarly, the link portion 120 includes an upper arm link 121 and a pair of forearm links 122 and 123, the link portion 130 includes an upper arm link 131 and a pair of forearm links 132 and 133, one end of each of the upper arm links 121 and 131 is turnably coupled to the base portion 101, and the end portion 102 is supported by respective other ends of the pairs of forearm links 122 and 123, and 132 and 133.

Each of the upper arm links 111, 121, and 131 extends radially outward from a center point on the base portion 101. Then, each of the upper arm links 111, 121, and 131 is pivotally supported on the base portion 101 in the vicinity of the lower end so as to be turnable in a vertical plane including the center point of the base portion 101. Referring to FIG. 3, the link portion 110 and the link portion 120 are arranged at an interval of 90 degrees, and the link portion 120 and the link portion 130 are arranged at an interval of 135 degrees with respect to the center point of the base portion 101. Here, for convenience of explanation, the x-axis is set in the radial direction including the upper arm link 111, and the y-axis is set in the radial direction including the upper arm link 121.

The upper arm link 111 has one end connected with the output shaft of the actuator 141 mounted on the base portion 101, and turns so that the other end of the upper arm link 111 rises or falls when being driven to rotate by the actuator 141. Similarly, the upper arm link 121 and the upper arm link 131 have one ends connected respectively with the output shafts of the actuators 142 and 143 mounted on the base portion 101, and turn so that the other ends of each of the upper arm links 121 and 131 moves up and down when being driven to rotate by the actuators 142 and 143. Accordingly, by synchronously driving three actuators 141 to 143 to rotate, each of the link portions 110, 120, and 130 turns so that the tip (distal end) moves up and down, and as a result, the end portion 102 supported by the forearm links 112 and 113, 122 and 123, and 132 and 133 can be translated to any position in three-dimensional space.

Note that each of the actuators 141 to 143 may include an encoder that detects the rotation position of the output shaft (or each of the link portions 110, 120, and 130 coupled to the output shaft), a torque sensor that detects external torque applied to the output shaft via each of the link portions 110, 120, and 130, or the like built therein.

The parallel link device 100 according to the present embodiment is characterized in that additional link mechanism portions for each driving the RCM structure portion 200 mounted on the end portion 102 are added to the two link portions 110 and 120. Two link portions 110 and 120 each have one degree of freedom to drive the tip portion. Accordingly, the parallel link device 100 as a whole can have a total of five-degree-of-freedom structure of three translational degrees of freedom and two rotational degrees of freedom.

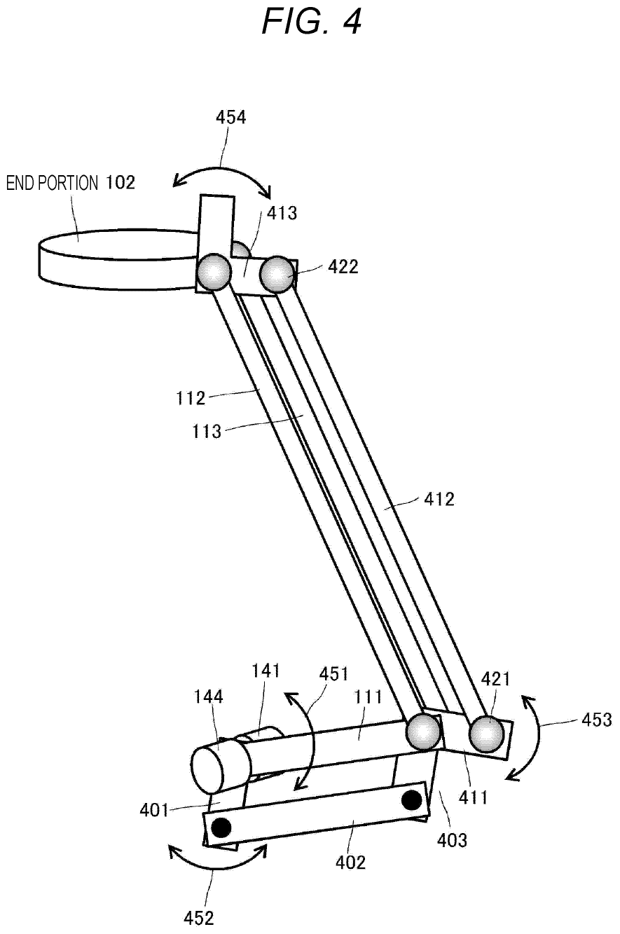

FIG. 4 schematically illustrates the structure of an additional link mechanism portion equipped in addition to the link portion 110. The structure and action of the additional link mechanism portion equipped in addition to the link portion 110 will be described with reference to FIG. 4.

Links 401, 402, and 403 that configure a four-section link together with the upper arm link 111 are added to the upper arm link 111. Furthermore, links 411, 412, and 413 are also added to the pair of forearm links 112 and 113 so as to configure a four-section link.

As described above, the upper arm link 111 is connected with the output shaft of the actuator 141 and turns in the direction indicated by the reference number 451 in FIG. 4. When the upper arm link 111 swings in the rotation direction 451, the tip of the link portion 110 rises or falls. On the other hand, as a four-section link, the upper arm link 111 corresponds to a stator section, the link 401 corresponds to a driver section, the link 402 corresponds to a coupler section, and the link 403 corresponds to a follower section.

The actuator 144 arranged to face the actuator 141 turns the driver section 401 in the direction indicated by the reference number 452 in FIG. 4. The turning motion of the driver section 401 is transmitted to the follower section 403 via the coupler section 402, and the follower section 403 swings in the direction indicated by the reference number 453 by substantially the same rotation angle.

The link 403 that acts as a follower section of the four-section link on the upper arm link 111 side is integrated with the link 411 that acts as a driver section of the four-section link on the forearm link 112 and 113 side. In the example illustrated in FIG. 4, a single structure (rigid body) is configured in which one end portion of an L-shaped plate is the link 403, and the other end portion is the link 411.

However, it is only required that the link 403 and the link 411 are configured to turn integrally, and it is not essential that the link 403 and the link 411 are configured as one structure. For example, the link 403 and the link 411 may be configured as separate members and may have a structural form firmly connected by a truss structure or the like.

When the actuator 141 is driven to rotate, the forearm links 112 and 113 coupled to the other end of the upper arm link 111 turn so as to rise or fall as described above. On the other hand, as a four-section link, the forearm links 112 and 113 correspond to stator sections, the link 411 corresponds to a driver section, the link 412 corresponds to a coupler section, and the link 413 corresponds to a follower section.

Furthermore, when the actuator 144 turns the link 401 as a driver section in the direction indicated by the reference number 452, the link 403 corresponding to the follower section swings in the direction indicated by the reference number 453 by substantially the same rotation angle as described above. Then, since the link 411 is integrated with the link 403, the rotation drive of the link 40 by the actuator 144 is also transmitted to the four-section link mechanism on the forearm link 112 and 113 side.

When the link 411 as a driver section turns integrally with the link 403, it is transmitted to the follower section 413 via the coupler section 412, and the follower section 413 swings in the direction indicated by the reference number 454 by substantially the same rotation angle. Note that it is desirable to use spherical joints for the connection portion 421 between the driver section 411 and one end of the coupler section 412, and the connection portion 422 between the other end of the coupler section 412 and the follower section 413 in consideration of tilt absorption.

The other end of the follower section 413 is coupled to the RCM structure portion 200 mounted on the end portion 102 (illustration is omitted in FIG. 4). Furthermore, the swing direction 454 shown in FIG. 4 coincides with the x direction in FIG. 3. Accordingly, the swing in the x direction indicated by the reference number 454 can be converted into rotation around the y-axis, so that rotation force around the y-axis is applied to the RCM structure portion 200 mounted on the end portion 102.

To summarize the structure illustrated in FIG. 4, the additional link mechanism portion added to the link portion 110 includes a four-section link mechanism configured by incorporating the upper arm link 111, and a four-section link mechanism configured by incorporating the forearm links 112 and 113, and is configured in a manner such that the follower section 403 on one four-section link mechanism side and the driver section 411 on the other four-section link mechanism side are integrated. Accordingly, the tip (or the end portion 102 coupled to the tip) of the link portion 110 can be translated by the actuator 141 arranged in the vicinity of the base of the link portion 110, while the other actuator 144 arranged in the vicinity of the base of the link portion 110 drives the four-section link mechanism having the upper arm link 111 incorporated therein, and the four-section link mechanism having the forearm links 112 and 113 incorporated therein transmits the driving force to the tip of the link portion 110, so as to realize remote rotation of the RCM structure portion 200 mounted on the end portion 102 around the y-axis. The additional link mechanism added to the link portion 110 can also be referred to as a transmission mechanism that transmits the driving force of the actuator 144 to the tip of the link portion 110 along the link portion 110.

Furthermore, although illustration and description of the additional link mechanism portion equipped in addition to the link portion 120 are omitted, the additional link mechanism portion acts with a similar configuration to and similarly to the additional link mechanism portion of the link portion 110. That is, a four-section link mechanism configured by incorporating the upper arm link 121, and a four-section link mechanism configured by incorporating the forearm links 122 and 123 are provided, and the follower section on one four-section link mechanism side and the driver section on the other four-section link mechanism side are integrated. Then, the tip (or the end portion 102 coupled to the tip) of the link portion 120 is translated by the actuator 142 arranged in the vicinity of the base of the link portion 120, and the other actuator 145 arranged in the vicinity of the base of the link portion 110 drives the four-section link mechanism so as to realize remote rotation of the RCM structure portion 200 mounted on the end portion 102. The additional link mechanism added to the link portion 120 can also be referred to as a transmission mechanism that transmits the driving force of the actuator 145 to the tip of the link portion 120 along the link portion 120.

Referring to FIG. 3, the link portion 110 and the link portion 120 are arranged at an interval of 90 degrees with respect to the center point of the base portion 101. Here, the swinging direction obtained at the tip of the additional link mechanism portion added to the link portion 120 by drive of the actuator 145 coincides with the y direction. Accordingly, the swing in the y direction by the link portion 120 can be converted into rotation around the x-axis, so that rotation force around the x-axis is applied to the RCM structure portion 200.

In short, the RCM structure in the parallel link device 100 according to the present embodiment is combined with the translation structure but can be driven independently of the translation structure, and it is possible to realize a structure in which all the actuators are mounted on the base portion.

Referring back to FIG. 1, the link portion 110 can give the RCM structure portion 200 the rotational degree of freedom indicated by the reference number 201 in the figure at the tip part thereof. The rotation direction 201 coincides with the rotation direction 454 shown in FIG. 4, that is, rotation around the y-axis. Furthermore, the link portion 120 can give the RCM structure portion 200 the rotational degree of freedom around the x-axis indicated by the reference number 202 in the figure at the tip part thereof. Accordingly, the parallel link device 100 can give the RCM structure portion 200 mounted on the end portion 102 the rotational degree of freedom of two orthogonal axes.

The parallel link device 100 as a whole can have a total of five-degree-of-freedom structure of three translational degrees of freedom and two rotational degrees of freedom. The three translational degrees of freedom of these allow the end portion 102 to be translated with respect to the base portion 101, and the two rotational degrees of freedom allow the RCM structure portion 200 mounted on the end portion 102 to remotely rotate around two axes.

Here, the configuration and operation of the RCM structure portion 200 will be described.

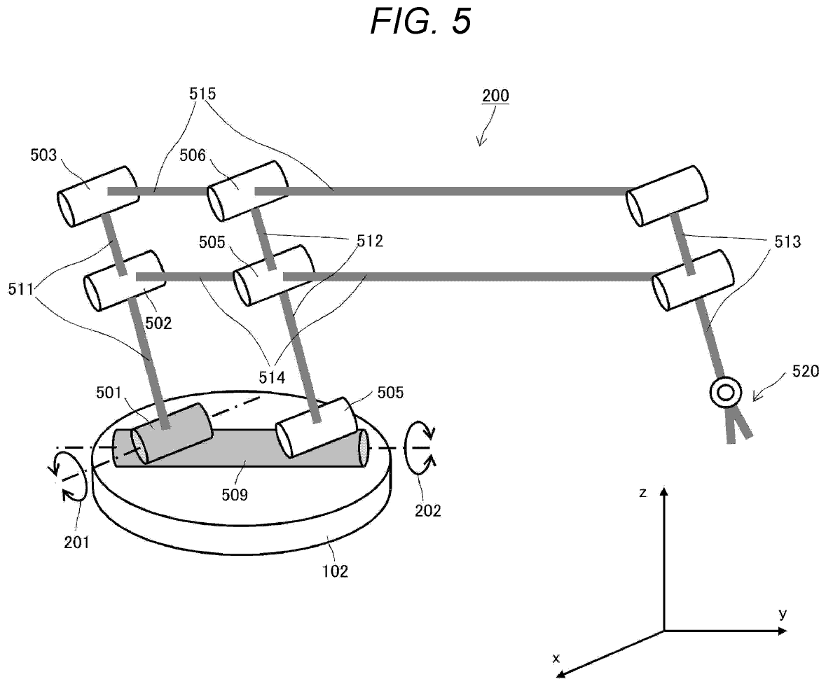

FIG. 5 illustrates the degree-of-freedom configuration of the RCM structure portion 200. However, the link part is shown by a thick line and the joint part is drawn with a cylinder (the rotation axis of each cylinder indicates the rotational degree of freedom of the corresponding joint) in the figure. Furthermore, for convenience, x, y, and z axes are set as shown in the figure.

Joints 501 to 508 are joints having a rotational degree of freedom around the x-axis. Furthermore, a joint 509 is a joint having a rotational degree of freedom around the y-axis. The RCM structure portion 200 is attached to the end portion 102 via the joint 509. Accordingly, the RCM structure portion 200 can change the posture around the y-axis with respect to the end portion 102 by driving the joint 509. The rotation force 202 (see FIG. 1) (or rotation force 454 in FIG. 4) from the parallel link device 100 side obtained using the additional link mechanism portion equipped in the link portion 110 can rotate the joint 509 around the y-axis. The rotation force 202 is obtained by driving the actuator 144 (not shown in FIG. 5) mounted in the vicinity of the base of the link portion 102 of the base portion 101. Accordingly, the RCM structure portion 200 can be referred to as an RCM structure in which the rotation center around the y-axis is arranged at a position away from the rotation center of the actuator 145.

Furthermore, the rotation force 201 (see FIG. 1) from the parallel link device 100 side obtained using the additional link mechanism portion equipped in the link portion 120 can rotate the joint 501 around the x-axis. Here, a four-section link mechanism is configured with links 511 to 515 coupled via the joints 501 to 508 that are turnable around the x-axis. Specifically, a four-section link mechanism is configured in which the link 511 is a driver section, the end portion 102 is a stator section, the link 514 or 515 is a coupler section, and the link 512 or 513 is a following axis. Then, when the driver section 511 turns around the x-axis due to the swing of the tip of the additional link mechanism portion of the link portion 120 in the y direction, it is transmitted to the follower section 512 or 513 via the coupler section 514 or 515, and the follower section 512 or 513 swings by substantially the same rotation angle following the driver section 511.

For example, in a case where the parallel link device 100 is applied to a medical robot used for surgery, diagnosis, or examination, a medical instrument such as a surgical tool such as forceps, tweezers, or a cutting instrument, or a medical observation device such as a microscope or an endoscope (rigid endoscope such as laparoscope or arthroscopy, or flexible endoscope such as gastrointestinal endoscope or bronchoscope) is attached to the tip of the follower section 513 as an end effector 520. The posture of the follower section 513 or the end effector 520 is obtained by driving the actuators 144 and 145 (not shown in FIG. 5) respectively mounted in the vicinity of the bases of the link portions 101 and 102 on the base portion 101. Accordingly, the follower section 513 or the end effector 520 can be referred to as an RCM structure in which the rotation center is arranged at a position away from the rotation center of the actuator 144. Furthermore, an actuator for driving the end effector 520 such as opening and closing forceps (not shown in FIG. 5) may be mounted in the vicinity of the tip of the follower section 513.

To summarize the parallel link device 100 illustrated in FIGS. 1 to 5, a translation structure of the end portion 102 is realized by driving the link portions 110, 120, and 130 respectively using the actuators 141, 142, and 143 mounted on the base portion 101, while a mechanism that drives the tips of the link portions 110 and 120 respectively by the actuators 144 and 145 placed respectively on the bases of the link portions 110 and 120 is equipped so as to realize an RCM structure that can rotate a structure mounted on the end portion 102 around two axes. Then, the parallel link device 100 has a configuration in which all actuators that drive the RCM structure and the translation structure are mounted on the base portion 101 while the RCM structure and the translation structure are combined to be driven independently, and the end portion 102 on which the RCM structure is mounted can be made smaller and lighter.

When the RCM structure portion 200 on the end portion 102 is driven by the actuators 144 and 145 mounted on the base portion 101, note that the displacement amount is likely to generate a model error deviated from an ideal model due to bending or backlash in a case where only the link mechanism is provided. Therefore, an encoder may be mounted on the joint 501 or the joint 509 directly driven by the parallel link device 100 in the RCM structure portion 200, so that the posture of the RCM structure portion 200 is measured more accurately, and precision control is performed by reducing the influence of the model error. Furthermore, an inertial measurement unit (IMU) may be mounted on the RCM structure portion 200 so that actual acceleration or angular acceleration can be detected.

In actual control, the parallel link device 100 illustrated in FIGS. 1 to 5 can have a structure in which the translation of the end portion 102 and the rotation of the RCM structure portion 200 are completely independent. If a braking mechanism such as an electromagnetic brake is mounted on the actuators 141 to 143 for translation and the actuators 141 to 143 are fixed by the braking mechanism when translation is not used, it is possible to suppress the risk of accidental translation. Furthermore, when the translation position is determined, the actuators 141 to 143 are fixed so that electric power is not required, which also can be used to hold the own weight. Of course, the same applies to the actuators 144 and 145 for RCM, that is, if the actuators 144 and 145 are fixed by the braking mechanism when the RCM structure portion 200 is not rotated (remote rotation), it is possible to suppress the risk of accidental rotation. In a case where such a braking mechanism is applied to the medical robot described above, for example, the braking mechanism can prevent unnecessary motion during operation and is also useful for ensuring the safety of treatment. Furthermore, since no recovery time from unnecessary actions is generated, efficient treatment can be expected.

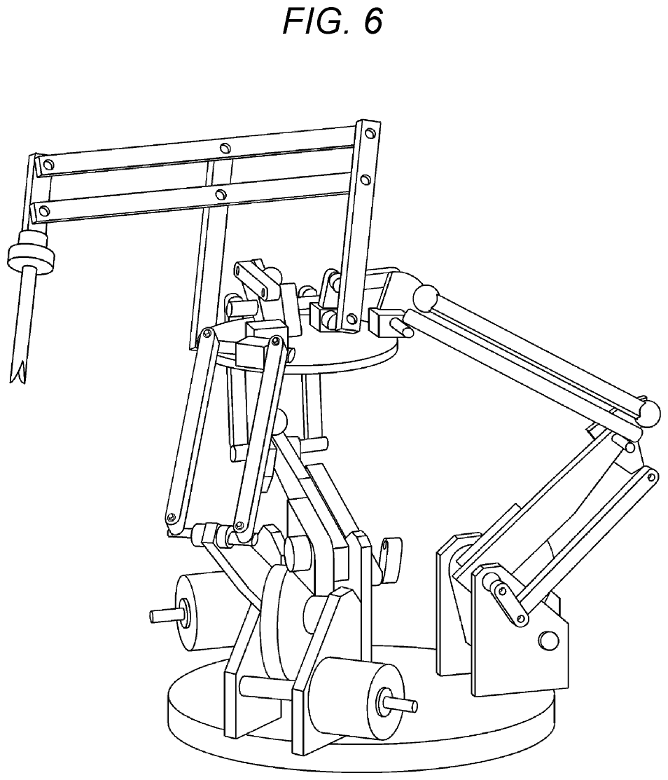

FIGS. 6 to 9 illustrate examples of the parallel link device 100 taking various postures. It should be understood from the figures that the end portion 102 translates with respect to the base portion 101 and the RCM structure portion 200 has the rotation center arranged at a position away from the rotation centers of the actuators 144 and 145 mounted on the base portion 101, and that the RCM structure portion 200 can be driven independently while being combined with the translation structure of the parallel link device 100.

Note that it is preferable to use a bearing as much as possible for each rotational sliding portion included in the parallel link device 100. However, regarding the RCM structure portion 200, it is also preferable to use a universal joint to transmit rotation from the link portion 110 or the link portion 120 in order to simplify the drive.

Furthermore, since the additional link mechanism portion equipped in addition to the link portion 110 or the link portion 120 swings with two degrees of freedom, it is also preferable to use a universal joint or a spherical bearing.

It is preferable that the structure such as the link member has a simple shape such as a rod shape or an L shape as much as possible in order to manufacture it at low cost.

The rotation axes of the actuator 141 and the actuator 144 arranged to face the vicinity of the base of the link portion 110 are assembled so as to coincide with each other. In order to ensure the accuracy, it is desirable to perform positioning in the same frame that can be completed by one machining. The same applies to the actuator 142 and the actuator 145 arranged to face the vicinity of the base of the link portion 120. It is preferable that the rotation shaft or each joint turnably connected between links has a double-sided structure in order to increase the rigidity.

To summarize the first embodiment, in a case where forceps are attached as the end effector 520 of the RCM structure portion 200, the parallel link device 100 can be rotated around the forceps tip, and the forceps can be translated completely independently of such rotation (RCM). Furthermore, it can be said that the parallel link device 100 is a composite parallel link structure that can be configured at low cost and compactly, since all the actuators 141 to 145 used for translation and rotation drive are mounted on the base portion 101.

The parallel link device 100 realizes a structure with low inertia by arranging all the actuators on the base portion 101 while serializing the translational and rotational parallel links. Since being configured with parallel links, a motor having a small output can be adopted as the actuator, which can improve safety and enable high-resolution force control. Furthermore, since the rotation mechanism in the upper stage and the translation mechanism in the lower stage can be structurally separated, the control calculation becomes easy, the operation frequency of each portion in the actual specifications is reduced, and the abrasion loss is reduced.

Although an additional link mechanism including a four-section link mechanism is placed on each of the link portions 110 and 120 in the parallel link device 100 illustrated in FIGS. 1 to 9 in order to remotely rotate the RCM structure portion 200 mounted on the end portion 102, note that the mechanism that drives the RCM structure portion 200 is not limited to a four-section link mechanism. For example, it is possible to achieve replacement with a mechanism or the like that transmits the driving force of the actuators 144 and 145 respectively to the tips of the link portions 110 and 120 using a belt or a gear.

Furthermore, although the link portion 110 and the link portion 120 to which additional link mechanisms are added are arranged at an interval of 90 degrees (see FIG. 3) in the parallel link device 100 illustrated in FIGS. 1 to 9, it is not necessarily limited to such an arrangement. For example, the link portion 110 and the link portion 120 may be arranged at an interval of approximately 60 degrees or approximately 120 degrees, and the link portion 120 and the link portion 130 not including an additional link mechanism may be arranged at an interval of approximately 120 degrees. However, from the viewpoint of efficiency of transmitting the driving force to the RCM structure portion 200 mounted on the end portion 102, reduction of the number of constituent members, or the like, it is desirable that the link portion 110 and the link portion 120 to which additional link mechanisms are added are arranged at an interval of 90 degrees.

Furthermore, in the parallel link device 100 illustrated in FIGS. 1 to 9, the link portion 120 to which an additional link mechanism is added and the link portion 130 not including an additional link mechanism are arranged at an interval of 135 degrees (see FIG. 3), which is a suitable arrangement in consideration of balance of forces when supporting the end portion 102 on which the RCM structure portion 200 is mounted, or reduction of backlash or bending. However, the intervals do not have to be exactly 135 degrees, and furthermore, the angles may be significantly different.

Furthermore, although each of the link portions 110, 120, and 130 is arranged at a position obtained by only rotation from the center point of the base portion 101 in the parallel link device 100 illustrated in FIGS. 1 to 9, it is not necessarily limited to such an arrangement. For example, any of the link portions may be closer to or farer from the center point of the base portion 101.

### Embodiment 2

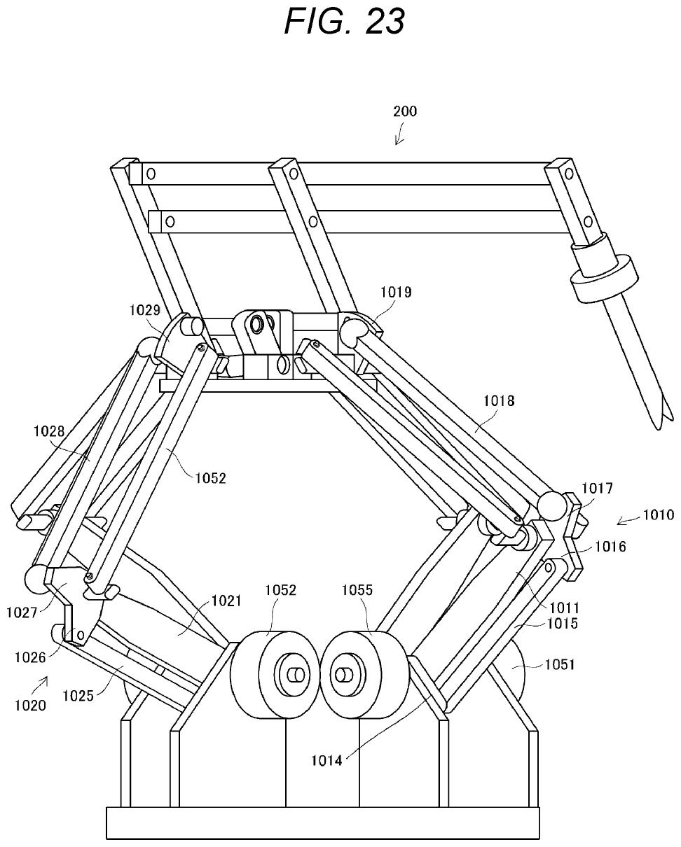

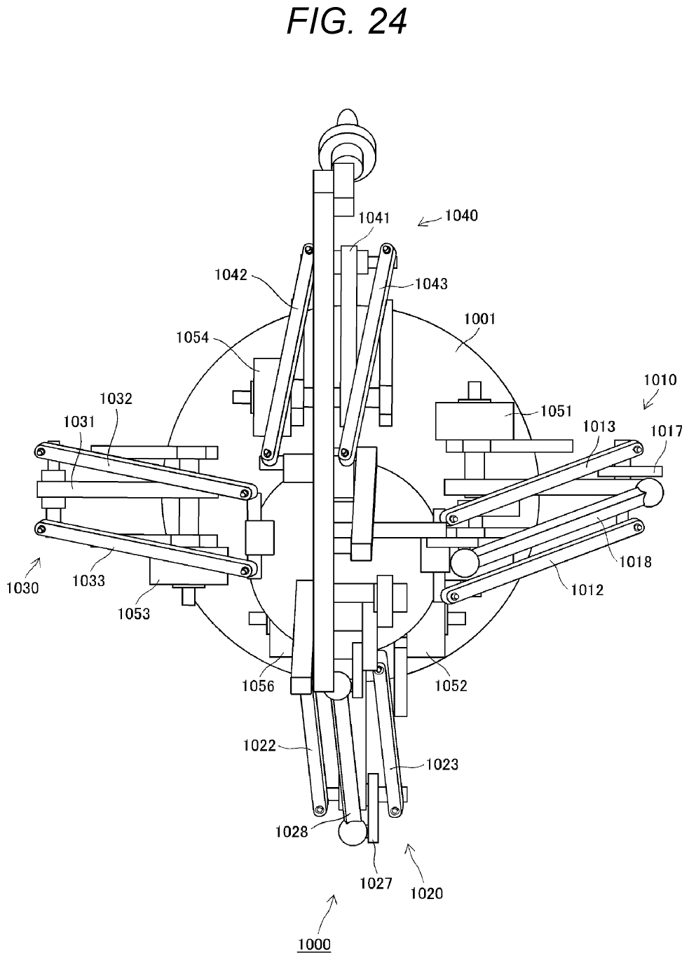

FIGS. 10, 23, and 24 illustrate a configuration example of a parallel link device 1000 according to the second embodiment. However, FIG. 10 shows a view of the parallel link device 1000 viewed obliquely, FIG. 23 shows a view of the parallel link device 1000 viewed from a side, and FIG. 24 shows a side of the parallel link device 1000 viewed from the opposite side to FIG. 10. The illustrated parallel link device 1000 includes a base portion 1001, an end portion 1002 that translates with respect to the base portion 1001, and four link portions 1010, 1020, 1030, and 1040 that are coupled to the base portion 1001 and support the end portion 1002. Furthermore, six actuators 1051 to 1056 for driving the link portions 1010, 1020, 1030, and 1040 are mounted on the base portion 1001.

Similarly to the parallel link device 100 (described above) according to the first embodiment, an RCM structure portion that realizes pivot motion around two axes may be mounted on the end portion 1002 of the parallel link device 1000 having a translation structure. For example, the RCM structure portion 200 illustrated in FIG. 5 can be directly mounted on the end portion 1002 of the parallel link device 1000.

The link portion 1010 includes an upper arm link 1011, and a pair of forearm links 1012 and 1013. One end of the upper arm link 1011 is turnably coupled to the base portion 1001, and the other end is turnably coupled to the pair of forearm links 1012 and 1013 via passive joints. Furthermore, the forearm links 1012 and 1013 support the end portion 1002 at the other ends. However, it is preferable to have a structure in which the upper arm link 1011 and each of the forearm links 1012 and 1013, and each of the forearm links 1012 and 1013 and the end portion 1002 are connected by, for example, spherical joints to absorb the inclination.

Similarly, the link portion 1020 includes an upper arm link 1021 and a pair of forearm links 1022 and 1023, the link portion 1030 includes an upper arm link 1031 and a pair of forearm links 1032 and 1033, and the link portion 1040 includes an upper arm link 1041 and a pair of forearm links 1042 and 1043. Then, one end of each of the upper arm links 1021, 1031, and 1041 is turnably coupled to the base portion 1001, and the end portion 1002 is supported by respective other ends of the pairs of forearm links 1022 and 1023, 1032 and 1033, and 1042 and 1043.

Each of the upper arm links 1011, 1021, 1031, and 1041 extends radially outward from a center point C on the base portion 1001. Then, each of the upper arm links 1011, 1021, and 1031 is pivotally supported by the base portion 1001 in the vicinity of the lower end so as to be turnable in a vertical plane including the center point C of the base portion 1001. As can be seen from FIG. 10, the link portions 1010, 1020, 1030, and 1040 are arranged at equal intervals of 90 degrees with respect to the center point C of the base portion 1001. Here, for convenience of explanation, the x-axis is set in the radial direction including the upper arm link 111, and the y-axis is set in the radial direction including the upper arm link 121.

The upper arm link 1011 has one end connected with the output shaft of the actuator 1051 mounted on the base portion 1001, and turns so that the other end of the upper arm link 111 moves up and down when being driven to rotate by the actuator 1051. Similarly, other upper arm links 1021, 1031, and 1041 also have one ends connected respectively with the output shafts of the actuators 1052, 1053, and 1054 mounted on the base portion 1001, and turn so that the other ends of the arm links 1021, 1031, and 1041 move up and down when being driven to rotate by the respective actuators 1052, 1053, and 1054.

Accordingly, by synchronously driving the four actuators 1051 to 1054 to rotate, each of the link portions 1010, 1020, 1030, and 1040 turns so that the tip (distal end) moves up and down, and as a result, the end portion 1002 supported by the forearm links 1012 and 1013, 1022 and 1023, 1032 and 1033, and 1042 and 1043 can be translated to any position in three-dimensional space. Note that each of the actuators 1051 to 1054 may include an encoder that detects the rotation position of the output shaft, a torque sensor that detects external torque applied to the output shaft, or the like built therein.

Since the parallel link device 1000 controls translation with three degrees of freedom by four actuators 1051 to 1054, it is possible to reduce backlash due to internal force as compared with the parallel link device 100 according to the first embodiment, and enables highly accurate action.

A parallel link structure including four or more links is already known in the industry. The main feature of the parallel link device 1000 according to the present embodiment is that an additional link mechanism portion for driving the RCM structure portion (not shown) mounted on the end portion 1002 is added to each of at least two link portions 1010 and 1020. Two link portions 1010 and 1020 each have one degree of freedom to drive the tip portion. Accordingly, the parallel link device 1000 can have a total of five-degree-of-freedom structure of three translational degrees of freedom and two rotational degrees of freedom.

As an additional link mechanism portion of the link portion 1010, links 1014, 1015, and 1016 that configure a four-section link together with the upper arm link 1011 are added, and links 1017, 1018, and 1019 are also added to the pair of forearm links 1012 and 1013 to configure a four-section link.

Here, the upper arm link 1011 corresponds to a stator section, the link 1014 corresponds to a driver section, the link 1015 corresponds to a coupler section, and the link 1016 corresponds to a follower section. Furthermore, the forearm links 1012 and 1013 correspond to stator sections, the link 1017 corresponds to a driver section, the link 1018 corresponds to a coupler section, and the link 1019 corresponds to a follower section. Then, the link 1016 that acts as a follower section of the four-section link on the upper arm link 1011 side is integrated with the link 1017 that acts as a driver section of the four-section link on the forearm link 1012 and 1013 side. The links 1016 and 1017 may have an integrated structure such as an L shape, for example, or may have a structural form firmly connected by a truss structure or the like.

The actuator 1055 arranged to face the actuator 1051 turns the driver section 1014. The turning motion of the driver section 1014 is transmitted to the follower section 1016 via the coupler section 1015, and the driver section 1017 integrated with the follower section 1016 swings by substantially the same rotation angle. Then, the follower section 1019 swings via the coupler section 1018. The tip of the follower section 1019 swings in the x direction in FIG. 10, which is converted into rotation around the y-axis, so that rotation force around the y-axis can be applied to the RCM structure portion (not shown) mounted on the end portion 1002.

Similarly, links 1024, 1025, and 1026 that configure a four-section link together with the upper arm link 1021 are added to the link portion 1020 as an additional link mechanism portion, and links 1027, 1028, and 1029 are also added to the pair of forearm links 1022 and 1023 to configure a four-section link. Then, the link 1026 that acts as a follower section of the four-section link on the upper arm link 1021 side is integrated with the link 1027 that acts as a driver section of the four-section link on the forearm link 1022 and 1023 side.

The actuator 1056 arranged to face the actuator 1052 turns the driver section 1024. The turning motion of the driver section 1024 is transmitted to the follower section 1026 via the coupler section 1025, and the driver section 1027 integrated with the follower section 1026 swings by substantially the same rotation angle. Then, the follower section 1029 swings via the coupler section 1028. The tip of the follower section 1029 swings in the y direction in FIG. 10, which is converted into rotation around the x-axis, so that rotation force around the x-axis can be applied to the RCM structure portion (not shown) mounted on the end portion 1002.

In a case where the RCM structure portion mounted on the end portion 1002 has a degree-of-freedom configuration as illustrated in FIG. 5, rotation force generated by the actuator 1055 equipped on the base of the link portion 1010 can be transmitted via the additional link mechanism portion to rotate the joint 509 around the y-axis. Furthermore, rotation force generated by the actuator 1056 equipped on the base of the link portion 1020 can be transmitted via the additional link mechanism portion to rotate the joint 501 around the x-axis.

Accordingly, the RCM structure portion can be referred to as an RCM structure in which the rotation centers around two axes of x and y are arranged at positions away from the rotation centers of the actuators 1055 and 1056.

The additional link mechanisms added to the link portions 1010 and 1020 can also be referred to as transmission mechanisms that transmit the driving force of the actuators 1055 and 1056 mounted on the base portion 1001 respectively to the tips of the link portions 1010 and 1020 respectively along the link portions 1010 and 1020.

Although other link portions 1030 and 1040 are drawn without additional link mechanism portions in FIG. 10, note that the link portions 1030 and 1040 may be equipped with additional link mechanism portions similar to the link portions 1010 and 1020.

In actual control, the parallel link device 1000 according to the present embodiment can have a structure in which the translation of the end portion 1002 and the rotation of the RCM structure portion (not shown) mounted on the end portion 1002 are completely independent. If a braking mechanism such as an electromagnetic brake is mounted on the actuators 1051 to 1054 for translation and the actuators 1051 to 1054 are fixed by the braking mechanism when translation is not used, it is possible to suppress the risk of accidental translation. Furthermore, when the translation position is determined, the actuators 1051 to 1054 are fixed so that electric power is not required, which also can be used to hold the own weight. Of course, the same applies to the actuators 1055 and 1056 for RCM, that is, if the actuators 1055 and 1056 are fixed by the braking mechanism when the RCM structure portion is not rotated (remote rotation), it is possible to suppress the risk of accidental rotation. In a case where such a braking mechanism is applied to the medical robot described above, for example, the braking mechanism can prevent unnecessary motion during operation and is also useful for ensuring the safety of treatment. Furthermore, since no recovery time from unnecessary actions is generated, efficient treatment can be expected.

The parallel link device 1000 realizes a structure with low inertia by arranging all the actuators on the base portion 1001 while serializing the translational and rotational parallel links. Since being configured with parallel links, a motor having a small output can be adopted as the actuator, which can improve safety and enable high-resolution force control. Furthermore, since the rotation mechanism in the upper stage and the translation mechanism in the lower stage can be structurally separated, the control calculation becomes easy, the operation frequency of each portion in the actual specifications is reduced, and the abrasion loss is reduced.

Note that it is preferable to use a bearing as much as possible for each rotational sliding portion included in the parallel link device 1000 illustrated in FIG. 10. However, regarding the RCM structure portion, it is also preferable to use a universal joint to transmit rotation from the link portion 1010 or the link portion 1020 in order to simplify the drive. Furthermore, since the additional link mechanism portion equipped in addition to the link portion 1010 or the link portion 1020 swings with two degrees of freedom, it is also preferable to use a universal joint or a spherical bearing. It is preferable that the structure such as the link member has a simple shape such as a rod shape or an L shape as much as possible in order to manufacture it at low cost.

Furthermore, although an additional link mechanism including a four-section link mechanism is placed on each of the link portions 1010 and 1020 in the parallel link device 1000 illustrated in FIG. 10 in order to remotely rotate the RCM structure portion mounted on the end portion 1002, the mechanism that drives the RCM structure portion to rotate is not limited to a four-section link mechanism. For example, it is possible to achieve replacement with a mechanism or the like that transmits the driving force of the actuators 1055 and 1056 respectively to the tips of the link portions 1010 and 1020 using a belt or a gear.

### Embodiment 3

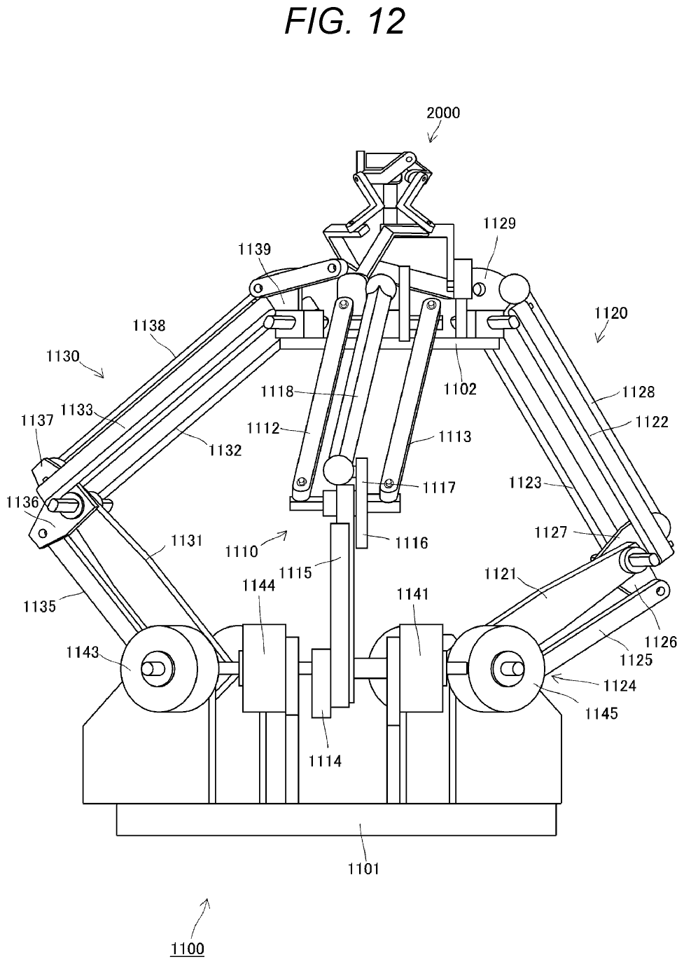

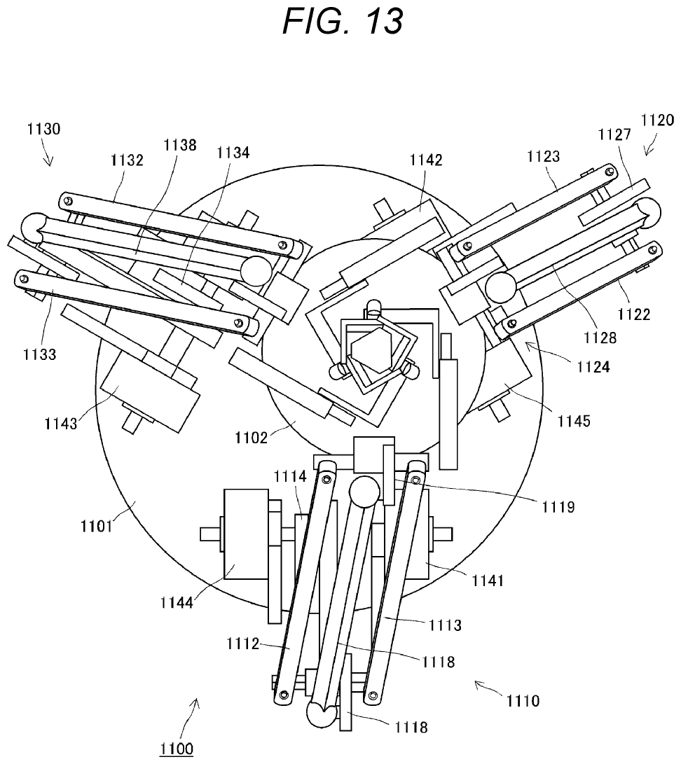

FIGS. 11 to 13 illustrate a configuration example of a parallel link device 1100 according to the third embodiment. However, FIG. 11 shows a view of the parallel link device 1100 viewed obliquely, FIG. 12 shows a view of the parallel link device 1100 viewed from the front, and FIG. 13 shows a view of the parallel link device 1100 viewed from above.

The illustrated parallel link device 1100 includes a base portion 1101, an end portion 1102 that translates with respect to the base portion 1101, and three link portions 1110, 1120, and 1130 that are coupled to the base portion 1101 and support the end portion 1102, and configures a delta type parallel link that generates three-translational-degree-of-freedom action. Furthermore, six actuators 1141 to 1146 for driving the link portions 1110, 1120, and 1130 are mounted on the base portion 1101. Although the actuators 1141 to 1146 are attached via a rib-shaped member projecting from the upper surface of the base portion 1101 in the examples illustrated in FIGS. 11 to 13, note that the actuators are not limited to a specific attachment structure. Moreover, an RCM structure portion 2000 having three rotational degrees of freedom is mounted on the end portion 1102.

The link portion 1110 includes an upper arm link 1111, and a pair of forearm links 1112 and 1113. One end of the upper arm link 1111 is turnably coupled to the base portion 1101, and the other end is turnably coupled to the pair of forearm links 1112 and 1113 via passive joints. Furthermore, the forearm links 1112 and 1113 support the end portion 1102 at the other ends. However, it is preferable to have a structure in which the upper arm link 1111 and each of the forearm links 1112 and 1113, and each of the forearm links 1112 and 1113 and the end portion 1102 are connected by, for example, spherical joints to absorb the inclination. Similarly, the link portion 1120 includes an upper arm link 1121 and a pair of forearm links 1122 and 1123, the link portion 1130 includes an upper arm link 1131 and a pair of forearm links 1132 and 1133, one end of each of the upper arm links 1121 and 1131 is turnably coupled to the base portion 1101, and the end portion 1102 is supported by respective other ends of the pairs of forearm links 1122 and 1123, and 1132 and 1133.

Each of the upper arm links 1111, 1121, and 1131 extends radially outward from a center point on the base portion 1101. Then, each of the upper arm links 1111, 1121, and 1131 is pivotally supported by the base portion 1101 in the vicinity of the lower end so as to be turnable in a vertical plane including the center point of the base portion 1101. Referring to FIG. 13, the link portions 1110, 1120, and 1130 are each arranged at intervals of 120 degrees with respect to the center point of the base portion 1001.

The upper arm link 1111 has one end connected with the output shaft of the actuator 1141 mounted on the base portion 1101, and turn so that the other end of the upper arm link 111 moves up and down when being driven to rotate by the actuator 1141. Similarly, the upper arm link 1121 and the upper arm link 1131 have one ends connected respectively with the output shafts of the actuators 1142 and 1143 mounted on the base portion 1101, and turn so that the other ends of the upper arm link 1121 and the upper arm link 1131 move up and down when being driven to rotate by the actuators 1142 and 1143. Accordingly, by synchronously driving three actuators 1141 to 1143 to rotate, each of the link portions 1110, 1120, and 1130 turns so that the tip (distal end) moves up and down, and as a result, the end portion 1102 supported by the forearm links 1112 and 1113, 1122 and 1123, and 1132 and 1133 can be translated to any position in three-dimensional space.

Note that each of the actuators 1141 to 1143 may include an encoder that detects the rotation position of the output shaft, a torque sensor that detects external torque applied to the output shaft via the link portions 1110, 1120, and 1130, or the like built therein.

The parallel link device 1100 according to the present embodiment is characterized in that an additional link mechanism portion for driving each rotation shaft of the RCM structure portion 2000 having three rotational degrees of freedom is added to each of all the three link portions 1110, 1120, and 1130. Accordingly, the parallel link device 1100 as a whole can have a total of three-degree-of-freedom structure of three translational degrees of freedom and three rotational degrees of freedom.

As an additional link mechanism portion of the link portion 1110, links 1114, 1115, and 1116 that configure a four-section link together with the upper arm link 1111 are added, and links 1117, 1118, and 1119 are also added to the pair of forearm links 1112 and 1113 to configure a four-section link.

Here, the upper arm link 1111 corresponds to a stator section, the link 1114 corresponds to a driver section, the link 1115 corresponds to a coupler section, and the link 1116 corresponds to a follower section. Furthermore, the forearm links 1112 and 1113 correspond to stator sections, the link 1117 corresponds to a driver section, the link 1118 corresponds to a coupler section, and the link 1119 corresponds to a follower section. Then, the link 1116 that acts as a follower section of the four-section link on the upper arm link 1111 side is integrated with the link 1117 that acts as a driver section of the four-section link on the forearm link 1112 and 1113 side. The links 1116 and 1117 may have an integrated structure such as an L shape, for example, or may have a structural form firmly connected by a truss structure or the like.

The actuator 1144 arranged to face the actuator 1141 turns the driver section 1114. The turning motion of the driver section 1114 is transmitted to the follower section 1116 via the coupler section 1115, and the driver section 1117 integrated with the follower section 1116 swings by substantially the same rotation angle. Then, the follower section 1119 swings via the coupler section 1118. The swinging motion of the tip of the follower section 1119 can be converted into rotation to apply rotation force around one axis of the RCM structure portion 2000 having three rotational degrees of freedom.

Similarly, links 1124, 1125, and 1126 that configure a four-section link together with the upper arm link 1121 are added to the link portion 1120 as an additional link mechanism portion, and links 1127, 1128, and 1129 are also added to the pair of forearm links 1122 and 1123 to configure a four-section link. Then, the link 1126 that acts as a follower section of the four-section link on the upper arm link 1121 side is integrated with the link 1127 that acts as a driver section of the four-section link on the forearm link 1122 and 1123 side.

The actuator 1145 arranged to face the actuator 1142 turns the driver section 1124. The turning motion of the driver section 1124 is transmitted to the follower section 1126 via the coupler section 1125, and the driver section 1127 integrated with the follower section 1126 swings by substantially the same rotation angle. Then, the follower section 1129 swings via the coupler section 1128. The swinging motion of the tip of the follower section 1129 can be converted into rotation to apply rotation force around another axis of the RCM structure portion 2000 having three rotational degrees of freedom.

Furthermore, similarly, links 1134, 1135, and 1136 that configure a four-section link together with the upper arm link 1131 are added to the link portion 1130 as an additional link mechanism portion, and links 1137, 1138, and 1139 are also added to the pair of forearm links 1132 and 1133 to configure a four-section link. Then, the link 1136 that acts as a follower section of the four-section link on the upper arm link 1131 side is integrated with the link 1137 that acts as a driver section of the four-section link on the forearm link 1132 and 1133 side.

The actuator 1146 arranged to face the actuator 1143 turns the driver section 1134. The turning motion of the driver section 1134 is transmitted to the follower section 1136 via the coupler section 1135, and the driver section 1137 integrated with the follower section 1136 swings by substantially the same rotation angle. Then, the follower section 1139 swings via the coupler section 1138. The swinging motion of the tip of the follower section 1139 can be converted into rotation to apply rotation force around other one axis of the RCM structure portion 2000 having three rotational degrees of freedom.

The additional link mechanisms added to the link portions 1110, 1120, and 1130 can also be referred to as transmission mechanisms that transmit the driving force of the actuators 1144, 1145, and 1146 mounted on the base portion 1101 respectively to the tips of the link portions 1110, 1120, and 1130 respectively along the link portions 1110, 1120, and 1130.

Next, the configuration of the RCM structure portion 2000 will be described.

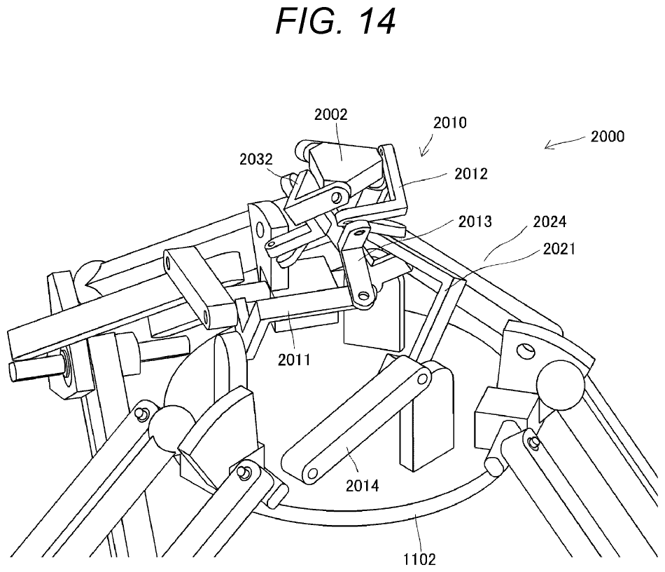

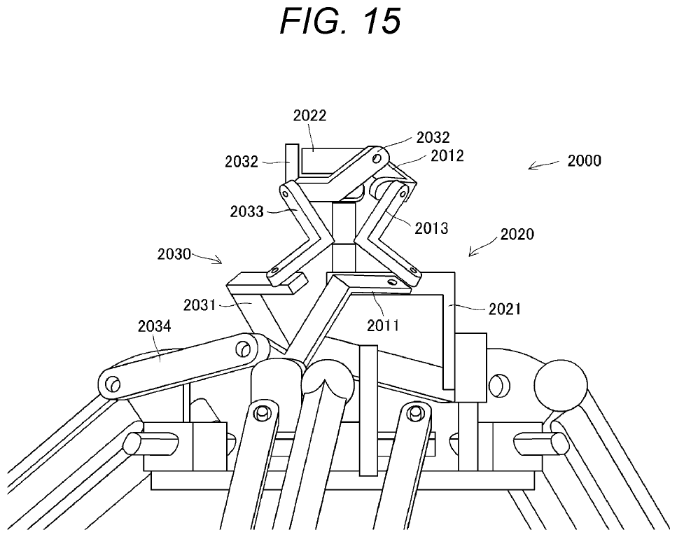

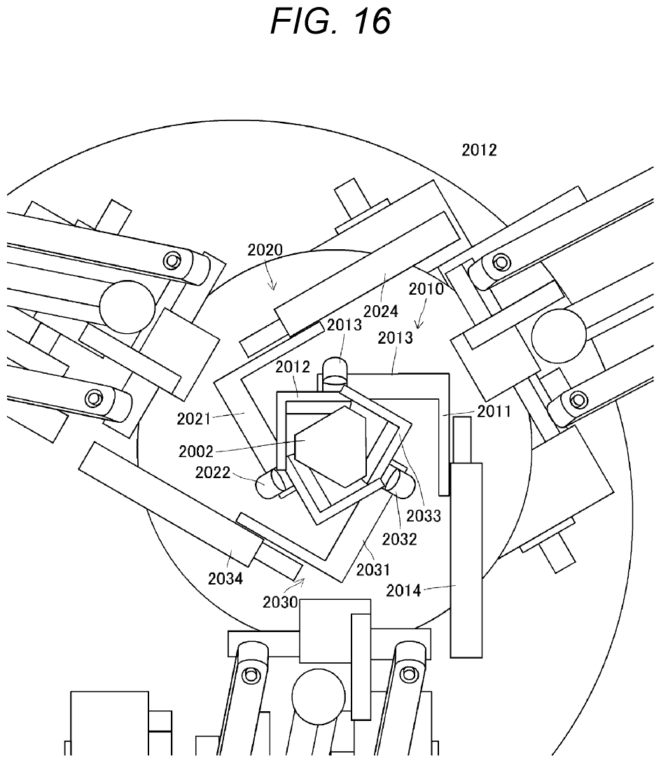

FIGS. 14 to 16 show the RCM structure portion 2000 in an enlarged manner. However, FIG. 14 shows a view of the RCM structure portion 2000 viewed obliquely, FIG. 15 shows a view of the RCM structure portion 2000 viewed from the front, and FIG. 16 shows a view of the RCM structure portion 2000 viewed from above.

The RCM structure portion 2000 has a parallel link structure in which the end portion 1102 is the base end side and three RCM link portions 2010, 2020, and 2030 support an RCM end portion 2002.

An RCM link portion 2010 is configured with an end link 2011 on the base end side, an end link 2012 on the tip side, that is, the RCM end portion 2002 side, and a central link 2013. The end links 2011 and 2012, and the central link 2013 each have an L shape. The end links 2011 and 2012 have one ends turnably coupled respectively to the end portion 1102 and the RCM end portion 2002. Then, both ends of the central link 2013 are rotatably coupled respectively to the other ends of the end links 2011 and 2012.

The end link 2011 is coupled to the vicinity of the tip of the follower section 1119 of the additional link mechanism portion added to the link portion 1110 via a link 2014 at one end on the base end side. Accordingly, when the actuator 1144 (described above) arranged in the vicinity of the base of the link portion 1110 is driven to rotate, it is transmitted by the additional link mechanism portion of the link portion 1110, so that the follower section 1119 swings, and thereby the end link 2011 turns around one end on the base end side as the central axis. Then, when the end link 2011 turns, the tip of the other end link 2012 turns so as to rise or fall, and changes the posture of the RCM end portion 2002 as a result.

Furthermore, the RCM link portion 2020 is configured with an end link 2021, an end link 2022, and a central link 2023 each having an L shape. The end links 2021 and 2022 have one ends rotatably coupled respectively to the end portion 1102 and the RCM end portion 2002. Then, both ends of the central link 2023 are rotatably coupled respectively to the other ends of the end links 2021 and 2022.

The end link 2021 is coupled to the vicinity of the tip of the follower section 1129 of the additional link mechanism portion added to the link portion 1120 via a link 2024 at one end on the base end side. When the actuator 1145 (described above) arranged in the vicinity of the base of the link portion 1120 is driven to rotate, it is transmitted by the additional link mechanism portion of the link portion 1120, so that the follower section 1129 swings, and thereby the end link 2021 turns around one end on the base end side as the central axis. Then, when the end link 2021 turns, the tip of the other end link 2022 turns so as to move up and down, and changes the posture of the RCM end portion 2002 as a result.

Furthermore, the RCM link portion 2030 is configured with an end link 2031, an end link 2032, and a central link 2033 each having an L shape. The end links 2031 and 2032 have one ends rotatably coupled respectively to the end portion 1102 and the RCM end portion 2002. Then, both ends of the central link 2033 are rotatably coupled respectively to the other ends of the end links 2031 and 2032.

The end link 2031 is coupled to the vicinity of the tip of the follower section 1139 of the additional link mechanism portion added to the link portion 1130 via a link 2034 at one end on the base end side. When the actuator 1146 (described above) arranged in the vicinity of the base of the link portion 1120 is driven to rotate, it is transmitted by the additional link mechanism portion of the link portion 1130, so that the follower section 1139 swings, and thereby the end link 2031 turns around one end on the base end side as the central axis R3. Then, when the end link 2031 turns, the tip of the other end link 2032 turns so as to move up and down, and changes the posture of the RCM end portion 2002 as a result.

In this way, the RCM link portions 2010, 2020, and 2030 can be driven respectively by three actuators 1144, 1145, and 1146 arranged on the base portion 1101 to change the posture of the uppermost RCM end portion 2002 around three axes, and the RCM structure portion 2000 has three rotational degrees of freedom.

When the RCM structure portion 2000 on the end portion 1102 is driven by the actuators 1144, 1145, and 1146 mounted on the base portion 1101, the displacement amount is likely to generate a model error deviated from an ideal model due to bending or backlash in a case where only the link mechanism is provided. Therefore, an encoder may be mounted on each of the RCM link portions 2010, 2020, and 2030 directly driven by the parallel link device 1100 in the RCM structure portion 2000, so that the posture of the RCM structure portion 2000 is measured more accurately, and precise control is performed by reducing the influence of the model error. Furthermore, an IMU may be mounted on the RCM structure portion 2000 so that actual acceleration or angular acceleration can be detected.

It can be said that the parallel link device 1100 according to the present embodiment has a structure in which an RCM structure portion 2000 including a parallel link structure having three rotational degrees of freedom is mounted on a delta type parallel link structure in the lower stage. The three translational degrees of freedom of the end portion 1102 by the delta type parallel link structure in the lower stage, and the three rotational degrees of freedom of the RCM structure portion 2000 mounted thereon can be made completely independent.

Note that the RCM structure portion 2000 mounted on the delta type parallel link structure in the lower stage of the parallel link device 1100 illustrated in FIGS. 11 to 13 is not necessarily limited to that illustrated in FIGS. 14 to 16. Various types of parallel link structures having three rotational degrees of freedom can be applied as the RCM structure portion 2000. For example, the link actuating device disclosed in Patent Document 4 or Patent Document 5 may be applied as the RCM structure portion 2000.

In actual control, the parallel link device 1100 according to the present embodiment can have a structure in which the translation of the end portion 1102 and the rotation of the RCM structure portion 2000 mounted on the end portion 1102 are completely independent. If a braking mechanism such as an electromagnetic brake is mounted on the actuators 1141 to 1143 for translation and the actuators 1141 to 1143 are fixed by the braking mechanism when translation is not used, it is possible to suppress the risk of accidental translation. Furthermore, when the translation position is determined, the actuators 1141 to 1143 are fixed so that electric power is not required, which also can be used to hold the own weight. Of course, the same applies to the actuators 1144 to 1146 for RCM, that is, if the actuators 1144 to 1146 are fixed by the braking mechanism when the RCM structure 2000 is not rotated (remote rotation), it is possible to suppress the risk of accidental rotation. In a case where such a braking mechanism is applied to the medical robot described above, for example, the braking mechanism can prevent unnecessary motion during operation and is also useful for ensuring the safety of treatment. Furthermore, since no recovery time from unnecessary actions is generated, efficient treatment can be expected.

The parallel link device 1100 realizes a structure with low inertia by arranging all the actuators on the base portion 1101 while serializing the translational and rotational parallel links. Since being configured with parallel links, a motor having a small output can be adopted as the actuator, which can improve safety and enable high-resolution force control. Furthermore, since the rotation mechanism in the upper stage and the translation mechanism in the lower stage can be structurally separated, the control calculation becomes easy, the operation frequency of each portion in the actual specifications is reduced, and the abrasion loss is reduced.

Note that it is preferable to use a bearing as much as possible for each rotational sliding portion included in the parallel link device 1100 illustrated in FIGS. 11 to 16. However, regarding the RCM structure portion 2000, it is also preferable to use a universal joint to transmit rotation from each of the link portions 1110, 1120, and 1130 in order to simplify the drive. Furthermore, since an additional link mechanism portion equipped in addition to each of the link portions 1110, 1120, and 1130 swings with two degrees of freedom, it is also preferable to use a universal joint or a spherical bearing. It is preferable that the structure such as the link member has a simple shape such as a rod shape or an L shape as much as possible in order to manufacture it at low cost.

Furthermore, although an additional link mechanism configured with a four-section link mechanism is placed at each of the link portions 1110, 1120, and 1130 in order to remotely rotate the RCM structure portion 2000 having three rotational degrees of freedom mounted on the end portion 1102 in the parallel link device 1100 illustrated in FIGS. 11 to 16, the mechanism that drives the RCM structure portion 2000 to rotate is not limited to a four-section link mechanism. For example, it is possible to achieve replacement with a mechanism or the like that transmits the driving force of the actuators 1144 to 1146 to the tip of each of the link portions 1110, 1120, and 1130 using a belt or a gear.

Furthermore, in the parallel link device 1100 illustrated in FIGS. 11 to 16, the link portions 1110, 1120, and 1130 to which additional link mechanisms are added are each arranged at intervals of 120 degrees. It can be said that this is a suitable arrangement in consideration of the balance of forces when supporting the end portion 1102 on which the RCM structure portion 2000 is mounted, or reduction of backlash or bending. However, the intervals do not have to be exactly 120 degrees, and furthermore, the angles may be significantly different. Furthermore, although each of the link portions 1110, 1120, and 1130 is arranged at a position obtained by only rotation from the center point of the base portion 1101, it is not necessarily limited to such an arrangement. For example, any of the link portions may be closer to or farer from the center point of the base portion 1101.

### Embodiment 4

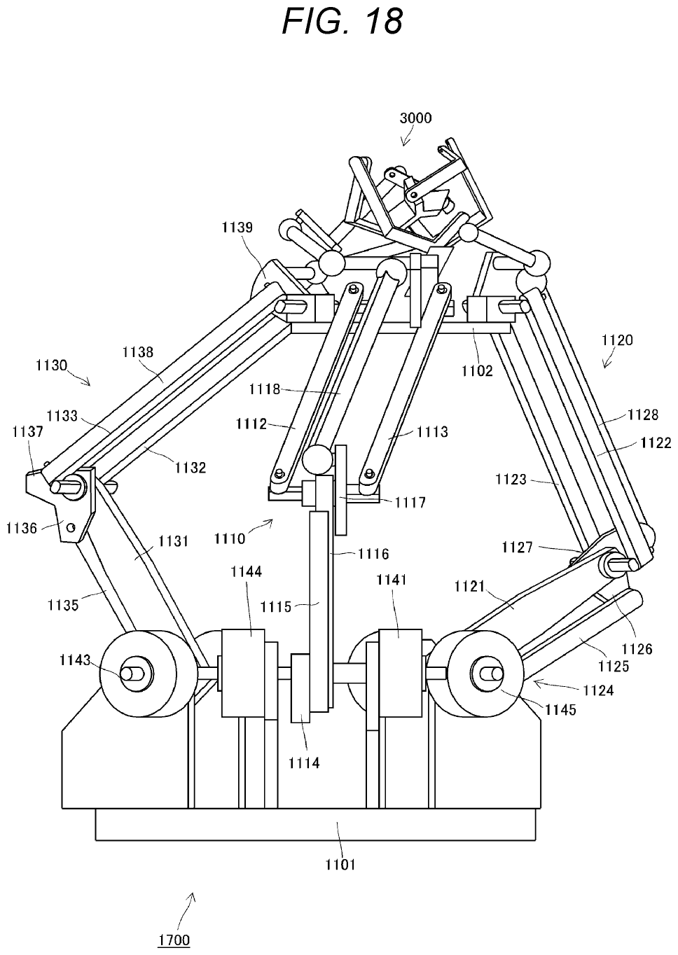

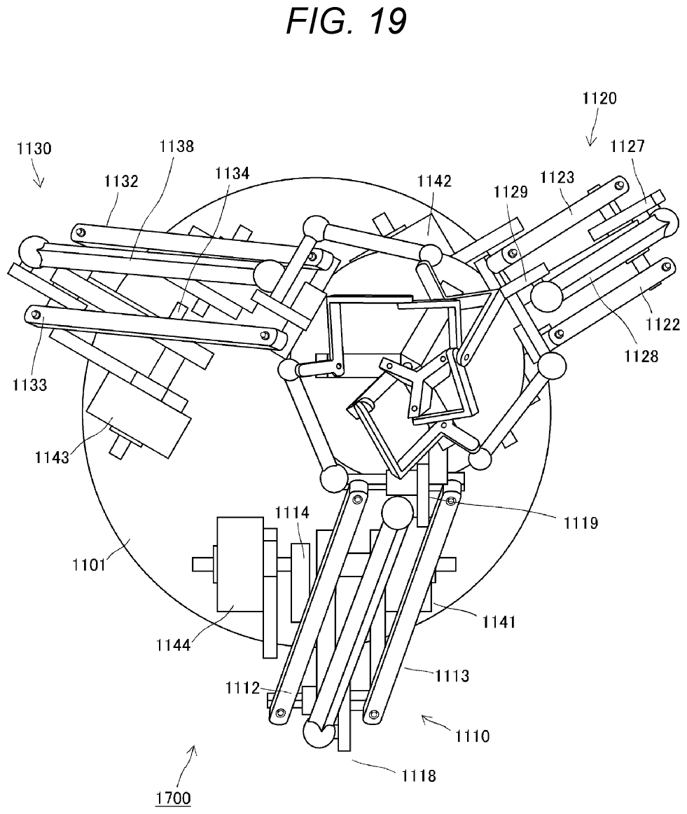

FIGS. 17 to 19 illustrate a configuration example of a parallel link device 1700 according to the fourth embodiment. However, FIG. 17 shows a view of the parallel link device 1700 viewed obliquely, FIG. 18 shows a view of the parallel link device 1700 viewed from the front, and FIG. 19 shows a view of the parallel link device 1700 viewed from above.

The illustrated parallel link device 1700 includes a base portion 1101, an end portion 1102 that translates with respect to the base portion 1101, and three link portions 1110, 1120, and 1130 that are coupled to the base portion 1101 and support the end portion 1102, and configures a delta type parallel link that generates three-translational-degree-of-freedom action. Furthermore, six actuators 1141 to 1146 for driving the link portions 1110, 1120, and 1130 are mounted on the base portion 1101. Although the actuators 1141 to 1146 are attached via a rib-shaped member projecting from the upper surface of the base portion 1101 in the examples illustrated in FIGS. 11 to 13, note that the actuators are not limited to a specific attachment structure. Moreover, an RCM structure portion 3000 that can be rotated by the above delta type parallel link is mounted on the end portion 1102.

The link portions 1110, 1120, and 1130 are each equipped with an additional link mechanism portion. The link portions 1110, 1120, and 1130 can be driven respectively by the actuators 1141, 1142, and 1143 to translate the end portion 1102. Furthermore, the additional link mechanism portions equipped respectively in the link portions 1110, 1120, and 1130 are driven respectively by the actuators 1146, 1147, and 1148 to drive the RCM structure portion 3000.

The additional link mechanisms added to the link portions 1110, 1120, and 1130 can also be referred to as transmission mechanisms that transmit the driving force of the actuators 1144, 1145, and 1146 mounted on the base portion 1101 respectively to the tips of the link portions 1110, 1120, and 1130 respectively along the link portions 1110, 1120, and 1130 (same as above). However, the base portion 1101 and the end portion 1102, and the delta type parallel link structure part configured with three link portions 1110, 1120, and 1130 are similar to those illustrated in FIGS. 11 to 13, and therefore detailed explanation is omitted here.

The RCM structure portion 3000 is a spherical parallel link device having three rotational degrees of freedom that includes three RCM link portions 3010, 3020, and 3030, each of which is configured to move on a spherical surface having a common center, and is also called “Agile eye”. The spherical parallel link device is characterized to have a wide range of motion and is driven at high speed and high acceleration.

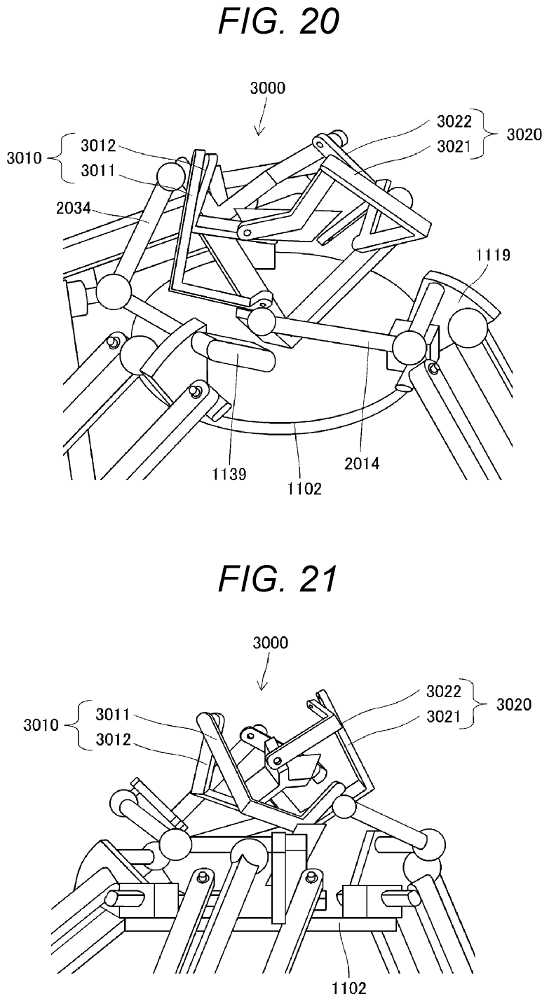

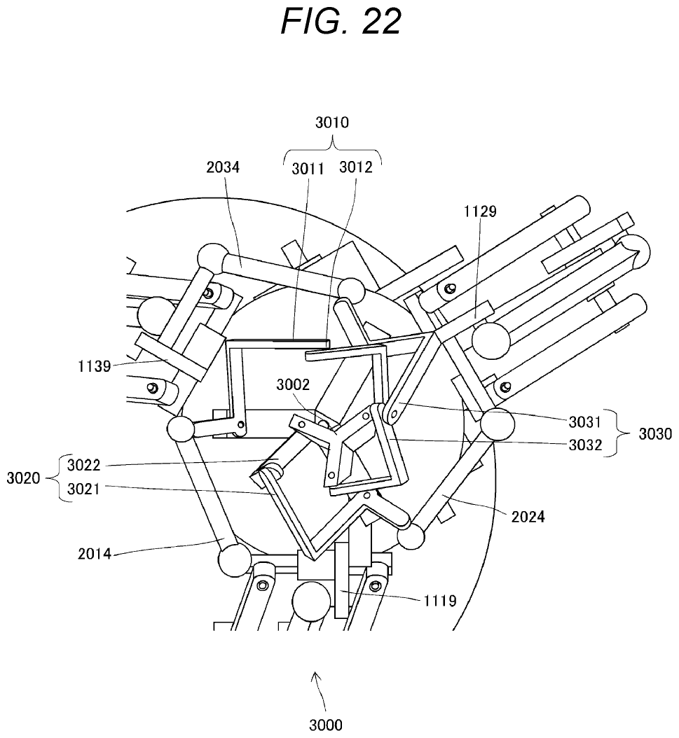

FIGS. 20 to 22 illustrate the RCM structure portion 3000 in an enlarged manner. However, FIG. 20 shows a view of the RCM structure portion 3000 viewed obliquely, FIG. 21 shows a view of the RCM structure portion 3000 viewed from the front, and FIG. 22 shows a view of the RCM structure portion 3000 viewed from above.

The RCM link portion 3010 is configured with an end link 3011 on the base end side and an end link 3012 on the tip side. The end link 3011 is coupled to the vicinity of the tip of a follower section 1119 of the additional link mechanism portion added to the link portion 1110 via a link 2014 at one end on the base end side. Furthermore, the RCM end portion 3002 is supported by the tip of the end link 3012.

When the actuator 1144 (described above) arranged in the vicinity of the base of the link portion 1110 is driven to rotate, it is transmitted by the additional link mechanism portion of the link portion 1110, so that the follower section 1119 swings, and thereby the end link 3011 turns around one end on the base end side as the central axis. Then, when the end link 3011 turns, the tip of the other end link 3012 turns around the common center described above, and changes the posture of the RCM end portion 3002 as a result.

Furthermore, the RCM link portion 3020 is configured with an end link 3021 on the base end side and an end link 3022 on the tip side. The end link 3021 is coupled to the vicinity of the tip of a follower section 1129 of the additional link mechanism portion added to the link portion 1120 via a link 2024 at one end on the base end side. Furthermore, the RCM end portion 3002 is supported by the tip of the end link 3022.

When the actuator 1145 (described above) arranged in the vicinity of the base of the link portion 1120 is driven to rotate, it is transmitted by the additional link mechanism portion of the link portion 1120, so that the follower section 1129 swings, and thereby the end link 3021 turns around one end on the base end side as the central axis. Then, when the end link 3021 turns, the tip of the other end link 3022 turns around the common center described above, and changes the posture of the RCM end portion 3002 as a result.

Furthermore, the RCM link portion 3030 is configured with an end link 3031 on the base end side and an end link 3032 on the tip side. The end link 3031 is coupled to the vicinity of the tip of a follower section 1139 of the additional link mechanism portion added to the link portion 1130 via the link 2024 at one end on the base end side. Furthermore, the RCM end portion 3002 is supported by the tip of the end link 3032.

When the actuator 1146 (described above) arranged in the vicinity of the base of the link portion 1130 is driven to rotate, it is transmitted by the additional link mechanism portion of the link portion 1130, so that the follower section 1139 swings, and thereby the end link 3031 turns around one end on the base end side as the central axis R3. Then, when the end link 3031 turns, the tip of the other end link 3022 turns around the common center described above, and changes the posture of the RCM end portion 3002 as a result.

In this way, the RCM link portions 3010, 3020, and 3030 can be driven respectively by three actuators 1144, 1145, and 1146 arranged on the base portion 1101 to rotate around the center of the above spherical surface of the uppermost RCM end portion 3002, and the RCM structure portion 3000 has three rotational degrees of freedom.

When the RCM structure portion 3000 on the end portion 1102 is driven by the actuators 1144, 1145, and 1146 mounted on the base portion 1101, the displacement amount is likely to generate a model error deviated from an ideal model due to bending or backlash in a case where only the link mechanism is provided. Therefore, an encoder may be mounted on each of the RCM link portions 3010, 3020, and 3030 directly driven by the parallel link device 1100 in the RCM structure portion 3000, so that the posture of the RCM structure portion 3000 is measured more accurately, and precise control is performed by reducing the influence of the model error. Furthermore, an IMU may be mounted on the RCM structure portion 3000 so that actual acceleration or angular acceleration can be detected.

It can be said that the parallel link device 1700 according to the present embodiment has a structure in which the RCM structure portion 3000 having a parallel link structure with three rotational degrees of freedom is mounted on the delta type parallel link structure in the lower stage. The three translational degrees of freedom of the end portion 1102 by the delta type parallel link structure in the lower stage, and the three rotational degrees of freedom of the RCM structure portion 3000 mounted thereon can be made completely independent.

Note that the RCM structure portion 3000 mounted on the delta type parallel link structure in the lower stage of the parallel link device 1700 illustrated in FIGS. 17 to 19 is not necessarily limited to that illustrated in FIGS. 20 to 22. Various types of parallel link structures having three rotational degrees of freedom can be applied as the RCM structure portion 3000. For example, the link mechanism disclosed in Patent Document 6 or the like may be applied as the RCM structure portion 3000.

In actual control, the parallel link device 1700 according to the present embodiment can have a structure in which the translation of the end portion 1102 and the rotation of the RCM structure portion 3000 mounted on the end portion 1102 are completely independent. If a braking mechanism such as an electromagnetic brake is mounted on the actuators 1141 to 1143 for translation and the actuators 1141 to 1143 are fixed by the braking mechanism when translation is not used, it is possible to suppress the risk of accidental translation. Furthermore, when the translation position is determined, the actuators 1141 to 1143 are fixed so that electric power is not required, which also can be used to hold the own weight. Of course, the same applies to the actuators 1144 to 1146 for RCM, that is, if the actuators 1144 to 1146 are fixed by the braking mechanism when the RCM structure 3000 is not rotated (remote rotation), it is possible to suppress the risk of accidental rotation. In a case where such a braking mechanism is applied to the medical robot described above, for example, the braking mechanism can prevent unnecessary motion during operation and is also useful for ensuring the safety of treatment. Furthermore, since no recovery time from unnecessary actions is generated, efficient treatment can be expected.

The parallel link device 1700 realizes a structure with low inertia by arranging all the actuators on the base portion 1101 while serializing the translational and rotational parallel links. Since being configured with parallel links, a motor having a small output can be adopted as the actuator, which can improve safety and enable high-resolution force control. Furthermore, since the rotation mechanism in the upper stage and the translation mechanism in the lower stage can be structurally separated, the control calculation becomes easy, the operation frequency of each portion in the actual specifications is reduced, and the abrasion loss is reduced.

Note that it is preferable to use a bearing as much as possible for each rotational sliding portion included in the parallel link device 1700 illustrated in FIGS. 17 to 22. However, regarding the RCM structure portion 3000, it is also preferable to use a universal joint to transmit rotation from each of the link portions 1110, 1120, and 1130 in order to simplify the drive. Furthermore, since an additional link mechanism portion equipped in addition to each of the link portions 1110, 1120, and 1130 swings with two degrees of freedom, it is also preferable to use a universal joint or a spherical bearing. It is preferable that the structure such as the link member has a simple shape such as a rod shape or an L shape as much as possible in order to manufacture it at low cost.

Furthermore, although an additional link mechanism configured with a four-section link mechanism is placed at each of the link portions 1110, 1120, and 1130 in order to remotely rotate the RCM structure portion 3000 having three rotational degrees of freedom mounted on the end portion 1102 in the parallel link device 1700 illustrated in FIGS. 17 to 22, the mechanism that drives the RCM structure portion 3000 to rotate is not limited to a four-section link mechanism. For example, it is possible to achieve replacement with a mechanism or the like that transmits the driving force of the actuators 1144 to 1146 to the tip of each of the link portions 1110, 1120, and 1130 using a belt or a gear.

Furthermore, in the parallel link device 1700 illustrated in FIGS. 17 to 22, the link portions 1110, 1120, and 1130 to which additional link mechanisms are added are each arranged at intervals of 120 degrees. It can be said that this is a suitable arrangement in consideration of the balance of forces when supporting the end portion 1102 on which the RCM structure portion 3000 is mounted, or reduction of backlash or bending. However, the intervals do not have to be exactly 120 degrees, and furthermore, the angles may be significantly different. Furthermore, although each of the link portions 1110, 1120, and 1130 is arranged at a position obtained by only rotation from the center point of the base portion 1101, it is not necessarily limited to such an arrangement. For example, any of the link portions may be closer to or farer from the center point of the base portion 1101.

### Embodiment 5

FIG. 25 schematically illustrates the functional configuration of a master-slave type robot system 2500. The robot system 2500 is configured with a master device 2510 operated by an operator, and a slave device 2520 remotely controlled from the master device 2510 according to operation by the operator. The master device 2510 and the slave device 2520 are interconnected via a wireless or wired network. In a case where the master-slave type robot system 2500 is applied to medical treatment such as surgery, or patient diagnosis or examination, the slave device 2520 holds the medical instrument, and the master device 2510 accepts operation input to the medical instrument by the operator. Then, the slave device 2520 receives the operation input to the medical instrument from the master device and operates the medical instrument.

The master device 2510 includes an operation unit 2511, a conversion unit 2512, a communication unit 2513, and an inner force sense presentation unit 2514.

The operation unit 2511 includes a master arm or the like for the operator to remotely operate the slave device 2520. The conversion unit 2512 converts the operation content performed by the operator on the operation unit 1411 into control information for controlling the drive of the slave device 2520 side (more specifically, a drive unit 2521 in the slave device 2520).

The communication unit 2513 is interconnected with the slave device 2520 side (more specifically, a communication unit 2523 in the slave device 1420) via a wireless or wired network. The communication unit 2513 transmits the control information outputted from the conversion unit 2512 to the slave device 2520.

On the other hand, the slave device 2520 includes the drive unit 2521, a detection unit 2522, and the communication unit 2523.

The drive unit 2521 of the slave device 2520 is assumed to be a parallel link device according to any one of the first to fourth embodiments described above. Furthermore, it is assumed that a medical instrument such as a surgical tool such as forceps, tweezers, or a cutting instrument, or a medical observation device such as a microscope or an endoscope (rigid endoscope such as laparoscope or arthroscopy, or flexible endoscope such as gastrointestinal endoscope or bronchoscope) is mounted on the RCM structure as an end effector. Then, the drive unit 2521 can drive each actuator arranged on the base portion of the parallel link device to translate the RCM structure or to remotely rotate a medical instrument mounted on the RCM structure independently of the translation movement.

The detection unit 2522 includes an encoder or a torque sensor built in each actuator arranged on the base portion, a sensor that measures the posture, acceleration, angular acceleration, or the like of the RCM structure, or the like. Furthermore, in a case where a gripping mechanism such as forceps is mounted on the RCM structure, the detection unit 2522 may include a sensor that detects the gripping force.

The communication unit 2523 is interconnected with the master device 2510 side (more specifically, the communication unit 2513 in the master device 2520) via a wireless or wired network. The above drive unit 2521 controls the drive of each actuator arranged on the base portion of the parallel link device according to control information from the master device 2510 side received by the communication unit 2523. Furthermore, the detection result by the above detection unit 2522 is sent from the communication unit 2523 to the master device 2510 side.

On the master device 2510 side, the inner force sense presentation unit 2514 carries out inner force sense presentation to the operator on the basis of the detection result received by the communication unit 2513 as feedback information from the slave device 2520. For example, a bilateral control method is applied to the robot system 2500, and the state of the slave device 2520 is fed back to the master device 2510 at the same time as the slave device 2520 is operated from the master device 2510.

The operator who operates the master device 2510 can recognize the contact force applied to the drive unit 2521 on the slave device 2520 side through the inner force sense presentation unit 2514. For example, in a case where the slave device 2520 is a medical robot, an operator such as a surgical operator can obtain a tactile sensation such as the response that acts on a medical instrument mounted on an RCM structure such as forceps, so as to properly adjust the thread operation, finish suturing completely, prevent invasion to living tissue, and work efficiently.

### INDUSTRIAL APPLICABILITY

The technology disclosed herein has been described above in detail with reference to specific embodiments. However, it is obvious that a person skilled in the art can make modifications or substitutions of the embodiments without departing from the gist of the technology disclosed herein.

Although the present specification has mainly described embodiments to which a delta type parallel link structure is applied, the gist of the technology disclosed herein is not limited thereto. Non-delta type parallel link structures, such as hexagonal parallel links capable of generating action of six degrees of freedom of translation and rotation or parallel links including four or more links are similarly applied, most actuators are mounted on the base portion, and a mechanism that drives the tips of two or more link portions is equipped, so as to realize an RCM structure that can be driven independently in combination with a translation structure.

Furthermore, it is assumed that a parallel link device proposed herein is applied to, for example, a medical robot used for surgery. In this case, an RCM structure is mounted on the end portion, and a medical instrument such as a surgical tool such as forceps, tweezers, or a cutting instrument, or a medical observation device such as a microscope or an endoscope (rigid endoscope such as laparoscope or arthroscopy, or flexible endoscope such as gastrointestinal endoscope or bronchoscope) is mounted at the tip of the RCM structure as an end effector and is used. Then, since the medical instrument can be remotely rotated independently of the translation movement of the end portion, a structure in which the medical instrument always passes through the position of the hole (e.g., trocar position) that is formed at the body of a patient during surgery is realized, and safety can be improved. Of course, the parallel link device proposed herein can be applied to various industrial applications other than medical treatment, such as industrial robots.

In short, the technology disclosed herein has been described in the form of exemplification, and the contents described herein should not be interpreted in a limited manner. To determine the gist of the technology disclosed herein, the claims should be taken into consideration.

Note that the technology disclosed herein can also have the following configurations.

(1) A parallel link device including:

an actuation unit that has a base portion, an end portion, and a plurality of link portions configured to couple the base portion and the end portion and drives the link portion using a first actuator mounted on the base portion to actuate the end portion with respect to the base portion; and

a transmission unit that transmits drive of a second actuator mounted on the base portion to a mechanism portion mounted on the end portion along each of at least two of the plurality of link portions.

(2) The parallel link device according to (1),

in which the mechanism portion has two rotational degrees of freedom, and

the transmission unit transmits drive of the second actuator along each of two of the plurality of link portions to rotate the mechanism portion around each axis.

(3) The parallel link device according to (2),

in which the two link portions that transmit drive of the second actuator are arranged at an interval of approximately 90 degrees.

(4) The parallel link device according to (3),

in which the actuation unit has a delta type parallel link structure, and

the two link portions and other one link portion are arranged at intervals of approximately 135 degrees.

(5) The parallel link device according to (1),

in which the mechanism portion has three rotational degrees of freedom, and

the transmission unit transmits drive of the second actuator along each of three of the plurality of link portions to rotate the mechanism portion around each axis.

(6) The parallel link device according to (1),

in which the mechanism portion includes a spherical parallel link having three rotational degrees of freedom configured to move on a spherical surface including a common center, and

the transmission unit transmits drive of the second actuator along each of three of the plurality of link portions to rotate the mechanism portion around each axis.

(7) The parallel link device according to (5) or (6),

in which the actuation unit has a delta type parallel link structure, and

the three link portions are respectively arranged at intervals of approximately 120 degrees.

(8) The parallel link device according to any one of (1) to (7), further including

a sensor that measures posture of the mechanism portion.

(9) The parallel link device according to (8),

in which the sensor includes an encoder that measures an angle at which the mechanism portion is rotated by the transmission unit.

(10) The parallel link device according to any one of (1) to (9), further including

a sensor that measures acceleration or angular acceleration of the mechanism portion.

(11) The parallel link device according to (10),

in which the sensor includes an inertia measuring device.

(12) The parallel link device according to any one of (1) to (11), further including

a communication unit that communicates with a master device,

in which the actuation unit drives at least one of the first actuator or the second actuator on the basis of control information received from the master device via the communication unit.

(13) The parallel link device according to any one of (8) to (11), further including

a communication unit that communicates with a master device,

in which the communication unit sends a detection signal of the sensor to the master device.

(14) A master-slave system including a master device and a slave device remotely operated by the master device,

in which the slave device includes:

an actuation unit that has a base portion, an end portion, and a plurality of link portions configured to couple the base portion and the end portion and drives the link portion using a first actuator mounted on the base portion to actuate the end portion with respect to the base portion; and

a transmission unit that transmits drive of a second actuator mounted on the base portion to a mechanism portion mounted on the end portion along each of at least two of the plurality of link portions.

(15) A medical master-slave system including:

a master device that accepts operation input to a medical instrument by an operator; and

a slave device that has a base portion, an end portion, and a plurality of link portions configured to couple the base portion and the end portion, holds the medical instrument on the end portion, and receives the operation input to the medical instrument from the master device to control the medical instrument,

in which the slave device includes:

an actuation unit that actuates the end portion with respect to the base portion; and

a transmission unit that transmits drive of a second actuator mounted on the base portion to a mechanism portion mounted on the end portion along each of at least two of the plurality of link portions.

### REFERENCE SIGNS LIST

100 Parallel link device
101 Base portion
102 End portion
110 Link portion
111 Upper arm link
112 and 113 Forearm link
120 Link portion
121 Upper arm link
122 and 123 Forearm link
130 Link portion
131 Upper arm link
132 and 133 Forearm link
141 to 143 Actuator (for translation movement)
144 and 145 Actuator (for RCM structure)
200 RCM structure portion
401 Link (driver section)
402 Link (coupler section)
403 Link (follower section)
411 Link (driver section)
412 Link (coupler section)
413 Link (follower section)
501 to 508 Joint (around x-axis)
509 Joint (around y-axis)
1100 Parallel link device
1101 Base portion
1102 End portion
1110 Link portion
1111 Upper arm link
1112 and 1113 Forearm link
1114 Link (driver section)
1115 Link (coupler section)
1116 Link (follower section)
1117 Link (driver section)
1118 Link (coupler)
1119 Link (follower section)
1120 Link portion
1121 Upper arm link
1122 and 1123 Forearm link
1124 Link (driver section)
1125 Link (coupler section)
1126 Link (follower section)
1127 Link (driver section)
1128 Link (coupler)
1129 Link (follower section)
1130 Link portion
1131 Upper arm link
1132 and 1133 Forearm link
1134 Link (driver section)
1135 Link (coupler section)
1136 Link (follower section)
1137 Link (driver section)
1138 Link (coupler)
1139 Link (follower section)
1141 to 1143 Actuator (for translation movement)
1144 and 1146 Actuator (for RCM structure)
2000 RCM structure portion
2010 RCM link portion
2011 End link (base end side)
2012 End link (RCM end side)
2013 Central link
2020 RCM link portion
2021 End link (base end side)
2022 End link (RCM end side)
2023 Central link
2030 RCM link portion
2031 End link (base end side)
2032 End link (RCM end side)
2033 Central link
2500 Robot system
2510 Master device
2511 Operation unit
2512 Conversion unit
2513 Communication unit
2514 Inner force sense presentation unit
2520 Slave device
2521 Drive unit
2522 Detection unit
2523 Communication unit

1. A parallel link device comprising:
an actuation unit that includes a base portion, an end portion, and a plurality of link portions configured to couple the base portion and the end portion and drives the link portion using a first actuator mounted on the base portion to actuate the end portion with respect to the base portion; and
a transmission unit that transmits drive of a second actuator mounted on the base portion to a mechanism portion mounted on the end portion along each of at least two of the plurality of link portions.

2. The parallel link device according to claim 1,
wherein the mechanism portion has two rotational degrees of freedom, and
the transmission unit transmits drive of the second actuator along each of two of the plurality of link portions to rotate the mechanism portion around each axis.

3. The parallel link device according to claim 2,
wherein two link portions that transmit drive of the second actuator are arranged at an interval of approximately 90 degrees.

4. The parallel link device according to claim 3,
wherein the actuation unit has a delta type parallel link structure, and
the two link portions and other one link portion are arranged at intervals of approximately 135 degrees.

5. The parallel link device according to claim 1,
wherein the mechanism portion has three rotational degrees of freedom, and
the transmission unit transmits drive of the second actuator along each of three of the plurality of link portions to rotate the mechanism portion around each axis.

6. The parallel link device according to claim 1,
wherein the mechanism portion includes a spherical parallel link having three rotational degrees of freedom configured to move on a spherical surface including a common center, and
the transmission unit transmits drive of the second actuator along each of three of the plurality of link portions to rotate the mechanism portion around each axis.

7. The parallel link device according to claim 5,
wherein the actuation unit has a delta type parallel link structure, and
the three link portions are respectively arranged at intervals of approximately 120 degrees.

8. The parallel link device according to claim 1, further comprising
a sensor that measures posture of the mechanism portion.

9. The parallel link device according to claim 8,
wherein the sensor includes an encoder that measures an angle at which the mechanism portion is rotated by the transmission unit.

10. The parallel link device according to claim 1, further comprising
a sensor that measures acceleration or angular acceleration of the mechanism portion.

11. The parallel link device according to claim 10,
wherein the sensor includes an inertia measuring device.

12. The parallel link device according to claim 1, further comprising
a communication unit that communicates with a master device,
wherein the actuation unit drives at least one of the first actuator or the second actuator on a basis of control information received from the master device via the communication unit.

13. The parallel link device according to claim 8, further comprising
a communication unit that communicates with a master device,
wherein the communication unit sends a detection signal of the sensor to the master device.

14. A master-slave system comprising a master device and a slave device remotely operated by the master device,
wherein the slave device includes:
an actuation unit that has a base portion, an end portion, and a plurality of link portions configured to couple the base portion and the end portion and drives the link portion using a first actuator mounted on the base portion to actuate the end portion with respect to the base portion; and
a transmission unit that transmits drive of a second actuator mounted on the base portion to a mechanism portion mounted on the end portion along each of at least two of the plurality of link portions.

15. A medical master-slave system comprising:
a master device that accepts operation input to a medical instrument by an operator; and
a slave device that includes a base portion, an end portion, and a plurality of link portions configured to couple the base portion and the end portion, holds the medical instrument on the end portion, and receives the operation input to the medical instrument from the master device to control the medical instrument,
wherein the slave device includes:
an actuation unit that actuates the end portion with respect to the base portion; and
a transmission unit that transmits drive of a second actuator mounted on the base portion to a mechanism portion mounted on the end portion along each of at least two of the plurality of link portions.
